(19) United States
(12) Patent Application Publication
(10) Pub. No.: US 2012/0225047 A1
(43) Pub. Date: Sep. 6, 2012

(54) NUTRIGENETIC BIOMARKERS FOR OBESITY AND TYPE 2 DIABETES

(76) Inventor: Jukka T. Salonen, Helsinki (FI)

(21) Appl. No.: 13/509,903

(22) PCT Filed: Nov. 16, 2010
(86) PCT No.: PCT/FI10/50923

§ 371 Date: May 15, 2012

(30) Foreign Application Priority Data
Nov. 16, 2009 (FI) 20096188

(51) Int. Cl. A61K 38/45; C40B 40/10; C07K 14/47; C40B 20/00; A61K 38/17; A61P 3/10; A61P 3/04; C40B 40/06; C12N 9/12

(52) U.S. Cl. 424/94.5; 506/16; 506/18; 530/350; 530/352; 435/194; 514/6.9; 514/4.8; 506/2

(57) ABSTRACT

The present invention discloses genes, SNP markers and haplotypes of susceptibility or predisposition to obesity, type 2 diabetes (T2D) and subdiagnosis of obesity and T2D and related medical conditions. Particularly, the present invention provides T2D and obesity associated markers from gene SUCLA2. Methods for diagnosis, prediction of clinical course and efficacy of treatments for T2D, obesity and related phenotypes using polymorphisms in the risk genes and other related biomarkers are also disclosed. Kits are also provided for the diagnosis, selecting treatment and assessing prognosis of obesity and T2D.

### BACKGROUND OF THE INVENTION

Obesity

Obesity is an excessive accumulation of energy in the form of body fat which impairs health. As the direct measurement of body fat is difficult, Body Mass Index (BMI), a simple ratio of weight to the square of height (kg/m²), is typically used to classify overweight (BMI>25) and obese (BMA>30) adults (Table 1). Consistent with this, the WHO has published international standards for classifying overweight and obesity in adults. The three major classes of obesity are monogenic, syndromic and polygenic obesity (or common obesity). Monogenic obesity is caused by a single dysfunctional gene and is typically familial, rare and severe form of obesity. Syndromic obesity is also rare and severe obesity form and there are about 30 Mendelian disorders, in which patients are clinically obese and have mental retardation, dysmorphic features and organ-specific developmental abnormalities. Polygenic obesity is a complex, multi-factorial chronic disease involving environmental (social and cultural), genetic, physiologic, metabolic, behavioral and psychological components and numerous genes seem to contribute to the obesity phenotype (Mutch and Clement, 2006).

TABLE 1
WHO Classification of Obesity

| WHO | Popular | BMI | |
|---|---|---|---|
| Classification | Description | (kg/m²) | Risk of co-morbidities |
| Underweight | Thin | <18.5 | Low (but risk of other |
| | | | clinical problems |
| | | | increased) |
| Normal range | Normal | 18.5-24.9 | Average |
| Overweight | | >25.0 | |
| Pre-obese | Overweight | 25-29.9 | Increased |
| Obese Class I | Obese | 30.0-34.9 | Moderate |
| Obese Class II | Obese | 35.0-39.9 | Severe |
| Obese Class III | Morbidly Obese | >40.0 | Very severe |

Although obesity is not a recent phenomenon as the historical roots of obesity can be traced back to 25,000 years ago, the epidemic of obesity is a recent global health issue across all age groups, especially in industrialized countries (American Obesity Association, 2006). According to WHO's estimate there are more than 300 million obese people (BMI>30) world-wide.

Today, for example almost 65% of adult Americans (about 127 million) are categorized as being overweight or obese. There is also evidence that obesity is increasing problem among children, for example in the USA, the percentage of overweight children (aged 5-14 years) has doubled in the last 30 years, from 15% to 32%.

The degree of health impairment of obesity is determined by three factors: 1) the amount of fat 2) the distribution of fat and 3) the presence of other risk factors. Obesity is the second leading cause of preventable death in the U.S. It affects all major bodily systems—heart, lung, muscle and bones—and is considered as a major risk factor for several chronic disease conditions, including coronary heart disease (CHD), type 2 diabetes mellitus (T2D), hypertension, cerebrovascular stroke, and cancers of the breast, endometrium, prostate and colon (Burton & Foster 1985).

The high prevalence of obesity, its significant contribution to morbidity and mortality of several common chronic diseases and lack of obesity related biomarkers and risk assessment tests show unmet medical need both for obesity related biomarkers as well as diagnostic methods and kits. The present invention provides a number of new relationships between various polymorphic alleles and common obesity. Obesity associated biomarkers disclosed in this invention provide the basis for improved risk assessment, more detailed diagnosis and prognosis of obesity.

Type 2 Diabetes

The term diabetes mellitus (DM) (ICD/10 codes E10-E14) describes several syndromes of abnormal carbohydrate metabolism that are characterized by hyperglycemia. It is associated with a relative or absolute impairment in insulin secretion, along with varying degrees of peripheral resistance to the action of insulin. The chronic hyperglycemia of diabetes is associated with long-term damage, dysfunction, and failure of various organs, especially the eyes, kidneys, nerves, heart, and blood vessels (ADA, 2003). According to the new etiologic classification of DM, four categories are differentiated: type 1 diabetes (T1D), type 2 diabetes (T2D), other specific types, and gestational diabetes mellitus (ADA, 2003).

In T1D, formerly known as insulin-dependent (IDDM), the pancreas fails to produce the insulin which is essential for survival. This form develops most frequently in children and adolescents, but is being increasingly diagnosed later in life. T2D, formerly named non-insulin-dependent (NIDDM), results from the body's inability to respond properly to the action of insulin produced by the pancreas. T2D occurs most frequently in adults, but is being noted increasingly in adolescents as well (WHO, 2004). It is the commonest form of diabetes mellitus accounting for 90% of all cases worldwide.

Relationship Between Nutrition, Genes and Health

Inter-individual genetic variation is a critical determinant of differences in nutrient requirements. The commonest type of genetic variability is the single nucleotide polymorphism (SNP), a single base substitution within the DNA sequence. These occur roughly once every 200 to 300 nucleotides in the human genome. Several genetic polymorphisms of importance to nutrition have been identified. As more such links between polymorphisms and disease conditions are characterized, the scope for targeting dietary information and recommendations to specific subpopulations will increase.

Nutrigenetics aims to understand how the genetic makeup of an individual determines or contributes to their response to diet, and thus considers underlying genetic polymorphisms. It is the science of identifying and characterizing gene variants associated with differential responses to nutrients, and relating this variation to disease states. Nutrigenetics will yield critically important information that will assist clinicians and nutritionists in identifying the optimal diet for a given individual, i.e. personalized nutrition.

SNPs are important in explaining some of the variations in response to food components. Specific genetic polymorphisms in humans change their metabolic responses to diet and other therapies and can have an important effect on disease risk. Inter-individual genetic variation is also a crucial determinant of differences in nutrient requirements and tolerances to nutrients.

It is already apparent that there are many polymorphisms that influence risk of nutrition-related chronic diseases like obesity and type 2 diabetes. SNP analysis provides a molecular tool for investigating the role of nutrition in human health and disease, and their consideration in clinical, metabolic and epidemiological studies and genetic screening can contribute enormously to the definition of optimal diets.

The present invention is especially directed to genetic markers such as SNPs of gene SUCLA2. The prior art such as Feitosa et al. (Diabetes. 2009;58(suppl 1):A304) discloses that SUCLA2 is associated with waist/hip ratio and that there is strong evidence that SUCLA2 is involved in the complex genetic architecture of coronary heart disease. However, no disclosure of particular SNPs relating to T2D or obesity is found in Feitosa et al.

### SUMMARY OF THE INVENTION

This invention describes novel genes and markers which are associated with individual's response to a method of therapy such as a known food, functional or non-functional or diet or dietary pattern or small molecule medicine or a biological therapeutic product. It presents novel examples of nutrigenetics for common traits such as obesity, type 2 diabetes (T2D) and a T2D related condition.

This invention relates to genes and biomarkers associated with a response to a method of therapy in weight reduction and diabetes and their use in the treatment and prevention of obesity, T2D and a T2D related condition such as metabolic syndrome, insulin resistance, glucose intolerance, and T2D complications such as retinopathy, nephropathy or neuropathy, coronary heart disease, cerebrovascular disease, congestive heart failure, intermittent claudication or other manifestations of arteriosclerosis. The present invention provides novel genes and individual SNP markers associated with a response to antiobesity and antidiabetic foods, diets and other therapies. The invention further relates to physiological and biochemical routes and pathways related to these genes.

The present invention relates to previously unknown associations between various genes, loci and biomarkers, and obesity and T2D. The detection of these biomarkers provides novel methods and systems for risk assessment and diagnosis of obesity, which will also improve risk assessment, diagnosis and prognosis of obesity related conditions comprising type 2 diabetes, diabetic complications, coronary artery disease, myocardial infarction, stroke and hypertension.

The major application of the current invention is its use to predict an individual's response to a particular weight reducing or antidiabetic food/method of therapy. It is a well-known phenomenon that in general, patients do not respond equally to the same food or method of therapy. Much of the differences in the response to a given food are thought to be based on genetic and protein differences among individuals in certain genes and their corresponding pathways. Our invention defines the genes associated with a response to known method(s) of therapy in obesity, T2D and related conditions. Therefore, genes and gene variations which are the subject of current invention may be used as a nutrigenetic diagnostic to predict a response to a method of therapy and guide choice of method(s) of therapy for treating, preventing or ameliorating the symptoms, severity or progression of obesity and T2D or a T2D related condition in a given individual (“personalized nutrition”, “personalized prevention”).

Still another object of the invention is to provide a method for prediction of clinical course, and efficacy and safety of therapeutic method(s) with current weight reduction and antidiabetic foods and other therapies for T2D using polymorphisms in the genes associated with such response.

Another object of the invention is providing novel pathways to elucidate the presently unknown modes of action of known antiobesity and antidiabetic foods and diets. A major object of the invention are gene networks influencing individual's response to a method of therapy with insulin secretors or insulin sensitizers or insulin are presented. Such gene networks can be used for other methods of the invention comprising diagnostic methods for prediction of the response to a particular food, the efficacy and safety of a particular food described herein and the treatment methods described herein.

Kits are also provided for the selection, prognosis and monitoring of the method of therapy for obesity and T2D. Better means for identifying those individuals who will benefit more from the selected method of therapy for obesity or T2D due to the better response and long-term glycemic control and fewer adverse effects should lead to better preventive and treatment regimens. Nutrigenetic information may be used to assist physician in choosing method of therapy for the particular patient (“personalized medicine”).

In summary, the invention helps meet the unmet medical needs and promotes public health in at least two major ways: 1) it provides novel means to predict individual's response and evaluate safety and efficiency of a selected method of therapy with known weight reducing or antidiabetic food or diet, as well as select the significant suitable alternative method of antiobesity or antidiabetic therapy for the individual (“personalized medicine”) and 2) it provides functional food and other therapeutic targets that can be used further to screen and develop functional foods and other therapeutic agents and therapies that can be used alone or in combination with the known antiobesity and antidiabetic therapies to treat, prevent or ameliorate the symptoms, severity or progression of obesity and T2D or a T2D related condition in a given individual.

Accordingly in a first aspect, the present invention provides methods and kits for diagnosing a susceptibility to high energy, carbohydrate or fat intake in an individual. The methods comprise the steps of: (i) obtaining a biological sample from the individual, and (ii) detecting in the biological sample the presence of one or more obesity and/or T2D associated biomarkers. These biomarkers may be SNP markers selected from Tables 6 through 17 of the invention or other biomarkers of the genes that they are associated with such as expressed RNA or protein or metabolites of the protein. The presence of obesity associated biomarkers in subject's sample is indicative of a susceptibility to high energy, carbohydrate or fat intake. The kits provided for diagnosing a susceptibility to high energy, carbohydrate or fat intake in an individual comprise wholly or in part protocol and reagents for detecting one or more biomarkers and interpretation software for data analysis and risk assessment. In one embodiment of this invention SNP markers being in linkage disequilibrium with one or more SNP markers of this invention are used in methods and kits for diagnosing a susceptibility to obesity. In other embodiment metabolites, expressed RNA molecules or expressed polypeptides, which are associated with one or more SNP markers of this invention are used in disclosed methods and kits.

In one typical embodiment, the biomarker information obtained from the methods diagnosing a susceptibility of an individual to high energy, carbohydrate or fat intake are combined with other information concerning the individual, e.g. results from blood measurements, clinical examination, questionnaires and/or interviews.

In one embodiment, the methods and kits of the invention are used in early diagnosis of obesity or T2D at or before onset, thus reducing or minimizing the debilitating effects of these conditions. In a preferred embodiment the methods and kits are applied in individuals who are free of clinical symptoms and signs of obesity and/or T2D, but have family history of obesity and/or T2D or in those who have multiple risk factors for obesity.

In a second aspect, the present invention provides methods and kits for molecular diagnosis i.e. determining a molecular subtype of obesity in an individual. In one preferred embodiment, molecular subtype of obesity in an individual is determined to provide information of the molecular etiology of obesity. When the molecular etiology is known, better diagnosis and prognosis of obesity can be made and efficient and safe therapy for treating obesity in an individual can be selected on the basis of this subtype information. For example, the food or other therapy that is likely to be effective, can be selected without trial and error. In other embodiment, biomarker information obtained from methods and kits for determining molecular subtype of obesity in an individual is for monitoring the effectiveness of obesity treatment. In one embodiment, methods and kits for determining molecular subtype of obesity are used to select human subjects for clinical trials testing efficacy of obesity therapies. The kits provided for diagnosing a molecular subtype of obesity in an individual comprise wholly or in part protocol and reagents for detecting one or more biomarkers and interpretation software for data analysis and obesity molecular subtype assessment.

### BRIEF DESCRIPTION OF THE DRAWINGS

FIG. 1. Linear regression between carbohydrate intake and BMI in genotypes of RS11792803.

FIG. 2. Linear regression between glycemic load and BMI for RS2847666.

FIG. 3. Linear regression between carbohydrate intake and WHR in RS10833641 genotypes.

FIG. 4. Linear regression between glycemic load and WHR in RS17023900 genotypes.

FIG. 5. Linear regression between glycemic index and WHR in RS3731572 genotypes.

FIG. 6. Linear regression between soluble carbohydrate intake (g/d) and BMI in RS16884072 A/G and G/G genotypes .

### DETAILED DESCRIPTION OF THE INVENTION

The present invention relates to previously unknown associations between high energy, carbohydrate or fat intake, obesity and various biomarkers. These novel obesity biomarkers provide basis for novel methods and kits for risk assessment and diagnosis of obesity and obesity related conditions.

A “biomarker” in the context of the present invention refers to a SNP marker disclosed in Tables 6 through 17 or to a polymorphism which is in linkage disequilibrium with one or more disclosed SNP markers, or to an organic biomolecule which is related to a SNP marker set forth in Tables 6 through 17 and which is differentially present in samples taken from subjects (patients) being obese compared to comparable samples taken from subjects who are non-obese (BMI<30). An “organic biomolecule” refers to an organic molecule of biological origin comprising steroids, amino acids, nucleotides, sugars, polypeptides, polynucleotides, complex carbohydrates and lipids. A biomarker is differentially present between two samples if the amount, structure, function or biological activity of the biomarker in one sample differs in a statistically significant way from the amount, structure, function or biological activity of the biomarker in the other sample.

A “haplotype,” as described herein, refers to a combination of genetic markers (“alleles”). A haplotype can comprise two or more alleles and the length of a genome region comprising a haplotype may vary from few hundred bases up to hundreds of kilobases. As it is recognized by those skilled in the art the same haplotype can be described differently by determining the haplotype defining alleles from different nucleic acid strands. E.g. the haplotype GGC defined by the SNP markers rs3936203, rs10933514 and rs4630763 of this invention is the same as haplotype rs3936203, rs10933514, and rs4630763 (CCG) in which the alleles are determined from the other strand, or haplotype rs3936203, rs10933514, and rs4630763 (CGC), in which the first allele is determined from the other strand. The haplotypes described herein are differentially present in individuals with obesity than in individuals without obesity. Therefore, these haplotypes have diagnostic value for risk assessment, diagnosis and prognosis of obesity in an individual. Detection of haplotypes can be accomplished by methods known in the art used for detecting nucleotides at polymorphic sites. Haplotypes found more frequently in obese individuals (risk increasing haplotypes) as well as haplotypes found more frequently in non-obese individuals (risk reducing haplotypes) have predictive value for predicting susceptibility to obesity in an individual.

A nucleotide position in genome at which more than one sequence is possible in a population, is referred to herein as a “polymorphic site” or “polymorphism”. Where a polymorphic site is a single nucleotide in length, the site is referred to as a SNP. For example, if at a particular chromosomal location, one member of a population has an adenine and another member of the population has a thymine at the same position, then this position is a polymorphic site, and, more specifically, the polymorphic site is a SNP. Polymorphic sites may be several nucleotides in length due to insertions, deletions, conversions or translocations. Each version of the sequence with respect to the polymorphic site is referred to herein as an “allele” of the polymorphic site. Thus, in the previous example, the SNP allows for both an adenine allele and a thymine allele.

Typically, a reference nucleotide sequence is referred to for a particular gene e.g. in NCBI databases (www.ncbi.nlm.nih.gov). Alleles that differ from the reference are referred to as “variant” alleles. The polypeptide encoded by the reference nucleotide sequence is the “reference” polypeptide with a particular reference amino acid sequence, and polypeptides encoded by variant alleles are referred to as “variant” polypeptides with variant amino acid sequences. Nucleotide sequence variants can result in changes affecting properties of a polypeptide. These sequence differences, when compared to a reference nucleotide sequence, include insertions, deletions, conversions and substitutions: e.g. an insertion, a deletion or a conversion may result in a frame shift generating an altered polypeptide; a substitution of at least one nucleotide may result in a premature stop codon, amino acid change or abnormal mRNA splicing; the deletion of several nucleotides, resulting in a deletion of one or more amino acids encoded by the nucleotides; the insertion of several nucleotides, such as by unequal recombination or gene conversion, resulting in an interruption of the coding sequence of a reading frame; duplication of all or a part of a sequence; transposition; or a rearrangement of a nucleotide sequence, as described in detail above. Such sequence changes alter the polypeptide encoded by an obesity susceptibility gene. For example, a nucleotide change resulting in a change in polypeptide sequence can alter the physiological properties of a polypeptide dramatically by resulting in altered activity, distribution and stability or otherwise affect on properties of a polypeptide. Alternatively, nucleotide sequence variants can result in changes affecting transcription of a gene or translation of its mRNA. A polymorphic site located in a regulatory region of a gene may result in altered transcription of a gene e.g. due to altered tissue specificity, altered transcription rate or altered response to transcription factors. A polymorphic site located in a region corresponding to the mRNA of a gene may result in altered translation of the mRNA e.g. by inducing stable secondary structures to the mRNA and affecting the stability of the mRNA. Such sequence changes may alter the expression of an obesity susceptibility gene.

The SNP markers to which we have disclosed novel obesity associations in Tables 6 through 17 of this invention have been known in prior art with their official reference SNP (rs) ID identification tags assigned to each unique SNP by the National Center for Biotechnological Information (NCBI). Each rs ID has been linked to specific variable alleles present in a specific nucleotide position in the human genome, and the nucleotide position has been specified with the nucleotide sequences flanking each SNP. For example the SNP having rs ID rs4737191 is SNP in chromosome 8, and variable alleles are C and T.

Although the numerical chromosomal position of a SNP may still change upon annotating the current human genome build the SNP identification information such as variable alleles and flanking nucleotide sequences assigned to a SNP will remain the same. Those skilled in the art will readily recognize that the analysis of the nucleotides present in one or more SNPs set forth in Tables 6 through 17 of this invention in an individual's nucleic acid can be done by any method or technique capable of determining nucleotides present in a polymorphic site using the sequence information assigned in prior art to the rs IDs of the SNPs listed in Tables 6 through 17 of this invention. As it is obvious in the art the nucleotides present in polymorphisms can be determined from either nucleic acid strand or from both strands.

It is understood that the obesity associated SNP markers described in Tables 6 through 17 of this invention may be associated with other polymorphisms. This is because the SNP markers listed in Tables 6 through 17 are so called tagging SNPs (tagSNPs). TagSNPs are loci that can serve as proxies for many other SNPs. The use of tagSNPs greatly improves the power of association studies as only a subset of loci needs to be genotyped while maintaining the same information and power as if one had genotyped a larger number of SNPs. These other polymorphic sites associated with the SNP markers listed in Tables 6 through 17 of this invention may be either equally useful as obesity biomarkers or even more useful as causative variations explaining the observed obesity association of SNP markers of this invention.

The term “gene,” as used herein, refers to an entirety containing entire transcribed region and all regulatory regions of a gene. The transcribed region of a gene including all exon and intron sequences of a gene including alternatively spliced exons and introns so the transcribed region of a gene contains in addition to polypeptide encoding region of a gene also regulatory and 5′ and 3′ untranslated regions present in transcribed RNA. Each gene has been assigned a specific and unique nucleotide sequence by the scientific community. By using the name of a gene those skilled in the art will readily find the nucleotide sequences of the corresponding gene and it's encoded mRNAs as well as amino acid sequences of it's encoded polypeptides although some genes may have been known with other name(s) in the art.

In certain methods described herein, an individual who has increased risk for developing obesity is an individual in whom one or more obesity associated polymorphisms selected from Tables 6 through 17 of this invention are identified. In other embodiment also polymorphisms associated to one or more SNPs set forth in Tables 6 through 17 may be used in risk assessment of obesity. The significance associated with an allele or a haplotype is measured by an odds ratio. In a further embodiment, the significance is measured by a percentage. In one embodiment, a significant risk is measured as odds ratio of 0.8 or less or at least about 1.2, including by not limited to: 0.1, 0.2, 0.3, 0.4, 0.5, 0.6, 0.7, 0.8, 1.2, 1.3, 1.4, 1.5, 1.6, 1.7, 1.8, 1.9, 2.0, 2.5, 3.0, 4.0, 5.0, 10.0, 15.0, 20.0, 25.0, 30.0 and 40.0. In a further embodiment, a significant increase or reduction in risk is at least about 20%, including but not limited to about 25%, 30%, 35%, 40%, 45%, 50%, 55%, 60%, 65%, 70%, 75%, 80%, 85%, 90%, 95% and 98%. In a further embodiment, a significant increase in risk is at least about 50%. It is understood however, that identifying whether a risk is medically significant may also depend on a variety of factors such as subject's family history of obesity, previously identified glucose intolerance, hypertriglyceridemia, hypercholesterolemia, elevated LDL cholesterol, low HDL cholesterol, elevated BP, hypertension, cigarette smoking, lack of physical activity, and inflammatory components as reflected by increased C-reactive protein levels or other inflammatory markers.

“Probes” or “primers” are oligonucleotides that hybridize in a base-specific manner to a complementary strand of nucleic acid molecules. By “base specific manner” is meant that the two sequences must have a degree of nucleotide complementarity sufficient for the primer or probe to hybridize to its specific target. Accordingly, the primer or probe sequence is not required to be perfectly complementary to the sequence of the template. Non-complementary bases or modified bases can be interspersed into the primer or probe, provided that base substitutions do not inhibit hybridization. The nucleic acid template may also include “non-specific priming sequences” or “nonspecific sequences” to which the primer or probe has varying degrees of complementarity. Probes and primers may include modified bases as in polypeptide nucleic acids (Nielsen PE et al, 1991). Probes or primers typically comprise about 15, to 30 consecutive nucleotides present e.g. in human genome and they may further comprise a detectable label, e.g., radioisotope, fluorescent compound, enzyme, or enzyme co-factor. Probes and primers to a SNP marker disclosed in Tables 6 to 17 are available in the art or can easily be designed using the flanking nucleotide sequences assigned to a SNP rs ID and standard probe and primer design tools. Primers and probes for SNP markers disclosed in Tables 6 through 17 can be used in risk assessment as well as molecular diagnostic methods and kits of this invention.

The invention comprises polyclonal and monoclonal antibodies that bind to a polypeptide related to one or more obesity associated SNP markers set forth in Tables 6 through 17 of the invention. The term “antibody” as used herein refers to immunoglobulin molecules or their immunologically active portions that specifically bind to an epitope (antigen, antigenic determinant) present in a polypeptide or a fragment thereof, but does not substantially bind other molecules in a sample, e.g., a biological sample, which contains the polypeptide. Examples of immunologically active portions of immunoglobulin molecules include F(ab) and F(ab′) fragments which can be generated by treating the antibody with an enzyme such as pepsin. The term “monoclonal antibody” as used herein refers to a population of antibody molecules that are directed against a specific epitope and are produced either by a single clone of B cells or a single hybridoma cell line. Polyclonal and monoclonal antibodies can be prepared by various methods known in the art. Additionally, recombinant antibodies, such as chimeric and humanized monoclonal antibodies, comprising both human and non-human portions, can be produced by recombinant DNA techniques known in the art. Antibodies can be coupled to various enzymes, prosthetic groups, fluorescent materials, luminescent materials, bioluminescent materials, or radioactive materials to enhance detection.

An antibody specific for a polypeptide related to one or more obesity associated SNP markers set forth in Tables 6 through 17 of the invention can be used to detect the polypeptide in a biological sample in order to evaluate the abundance and pattern of expression of the polypeptide. Antibodies can be used diagnostically to monitor protein levels in tissue such as blood as part of a test predicting the susceptibility to obesity or as part of a clinical testing procedure, e.g., to, for example, determine the efficacy of a given treatment regimen.

“An obesity related condition” in the context of this invention comprises type 2 diabetes, coronary artery disease, myocardial infarction, stroke, hypertension, dyslipidaemias and metabolic syndrome. “A T2D related condition” in the context of this invention comprises metabolic syndrome, insulin resistance, glucose intolerance, and T2D complications such as retinopathy, nephropathy or neuropathy, coronary heart disease, cerebrovascular disease, congestive heart failure, intermittent claudication or another manifestation of arteriosclerosis. As Obesity is the most important risk factor and predursor of T2D, all examples and applications described in this invention concern, in addition to obesity, also T2D and T2D related conditions.

Diagnostic Methods and Test Kits

One major application of the current invention is diagnosing a susceptibility to obesity. The risk assessment methods and test kits of this invention can be applied to any healthy person as a screening or predisposition test, although the methods and test kits are preferably applied to high-risk individuals (subjects who have e.g. family history of obesity, type 2 diabetes or hypertension, or previous glucose intolerance or elevated level of any other obesity risk factor). Diagnostic tests that define genetic factors contributing to obesity might be used together with or independent of the known clinical risk factors to define an individual's risk relative to the general population. Better means for identifying those individuals susceptible for obesity should lead to better preventive and treatment regimens, including more aggressive management of the risk factors related to obesity and related diseases e.g. physicians may use the information on genetic risk factors to convince particular patients to adjust their life style e.g. to stop smoking, to reduce caloric intake and to increase exercise.

In one embodiment of the invention, diagnosing a susceptibility to obesity in a subject, is made by detecting one or more SNP markers disclosed in Tables 6 through 17 of this invention in the subject's nucleic acid. The presence of obesity associated alleles of the assessed SNP markers (and haplotypes) in individual's genome indicates subject's increased risk for obesity. The invention also pertains to methods of diagnosing a susceptibility to obesity in an individual comprising detection of a haplotype in an obesity risk gene that is more frequently present in an individual being obese (affected), compared to the frequency of its presence in a healthy non-obese individual (control), wherein the presence of the haplotype is indicative of a susceptibility to obesity. A haplotype may be associated with a reduced rather than increased risk of obesity, wherein the presence of the haplotype is indicative of a reduced risk of obesity. In other embodiment of the invention, diagnosis of susceptibility to obesity is done by detecting in the subject's nucleic acid one or more polymorphic sites being in linkage disequilibrium with one or more SNP markers and disclosed in Tables 6 through 17 of this invention. Diagnostically the most useful polymorphic sites are those altering the biological activity of a polypeptide related to one or more obesity associated SNP markers set forth in Tables 6 through 17. Examples of such functional polymorphisms include, but are not limited to frame shifts, premature stop codons, amino acid changing polymorphisms and polymorphisms inducing abnormal mRNA splicing. Nucleotide changes resulting in a change in polypeptide sequence in many cases alter the physiological properties of a polypeptide by resulting in altered activity, distribution and stability or otherwise affect the properties of a polypeptide. Other diagnostically useful polymorphic sites are those affecting transcription of a gene or translation of it's mRNA due to altered tissue specificity, due to altered transcription rate, due to altered response to physiological status, due to altered translation efficiency of the mRNA and due to altered stability of the mRNA. Thus presence of nucleotide sequence variants altering the polypeptide structure and/or expression rate of a gene related to one or more obesity associated SNP markers set forth in Tables 6 through 17 of this invention in individual's nucleic acid is diagnostic for susceptibility to obesity.

In diagnostic assays determination of the nucleotides present in one or more obesity associated SNP markers disclosed in this invention in an individual's nucleic acid can be done by any method or technique which can accurately determine nucleotides present in a polymorphic site. Numerous suitable methods have been described in the art (see e.g. Kwok P-Y, 2001; Syvanen A-C, 2001), these methods include, but are not limited to, hybridization assays, ligation assays, primer extension assays, enzymatic cleavage assays, chemical cleavage assays and any combinations of these assays. The assays may or may not include PCR, solid phase step, a microarray, modified oligonucleotides, labeled probes or labeled nucleotides and the assay may be multiplex or singleplex. As it is obvious in the art the nucleotides present in a polymorphic site can be determined from either nucleic acid strand or from both strands.

In another embodiment of the invention, a susceptibility to obesity is assessed from transcription products related to one or more obesity associated SNP markers set forth in Tables 6 through 17 of this invention. Qualitative or quantitative alterations in transcription products can be assessed by a variety of methods described in the art, including e.g. hybridization methods, enzymatic cleavage assays, RT-PCR assays and microarrays. A test sample from an individual is collected and the said transcription products are assessed from RNA molecules present in the test sample and the result of the test sample is compared with results from obese subjects (affected) and healthy non-obese subjects (control) to determine individual's susceptibility to obesity.

In another embodiment of the invention, diagnosis of a susceptibility to obesity is made by examining expression, abundance, biological activities, structures and/or functions of polypeptides related to one or more obesity associated SNP markers disclosed in Tables 6 through 17 of this invention. A test sample from an individual is assessed for the presence of alterations in the expression, biological activities, structures and/or functions of the polypeptides, or for the presence of a particular polypeptide variant (e.g., an isoform) related to one or more obesity associated SNP markers set forth in Tables 6 through 17 of this invention. An alteration can be, for example, quantitative (an alteration in the quantity of the expressed polypeptide, i.e., the amount of polypeptide produced) or qualitative (an alteration in the structure and/or function of a polypeptide i.e. expression of a mutant polypeptide or of a different splicing variant or isoform). Alterations in expression, abundance, biological activity, structure and/or function of a obesity susceptibility polypeptide can be determined by various methods known in the art e.g. by assays based on chromatography, spectroscopy, colorimetry, electrophoresis, isoelectric focusing, specific cleavage, immunologic techniques and measurement of biological activity as well as combinations of different assays. An “alteration” in the polypeptide expression or composition, as used herein, refers to an alteration in expression or composition in a test sample, as compared with the expression or composition in a control sample and an alteration can be assessed either directly from the polypeptide itself or it's fragment or from substrates and reaction products of said polypeptide. A control sample is a sample that corresponds to the test sample (e.g., is from the same type of cells), and is from an individual who is not affected by obesity. An alteration in the expression, abundance, biological activity, function or composition of a polypeptide related to one or more obesity associated SNP markers set forth in Tables 6 through 17 of this invention in the test sample, as compared with the control sample, is indicative of a susceptibility to obesity. In another embodiment, assessment of the splicing variant or isoform(s) of a polypeptide encoded by a polymorphic or mutant gene related to one or more obesity associated SNP markers set forth in Tables 6 through 17 of this invention can be performed directly (e.g., by examining the polypeptide itself), or indirectly (e.g., by examining the mRNA encoding the polypeptide, such as through mRNA profiling).

Yet in another embodiment, a susceptibility to obesity can be diagnosed by assessing the status and/or function of biological networks and/or metabolic pathways related to one or more obesity associated SNP markers disclosed in Tables 6 through 17. Status and/or function of a biological network and/or a metabolic pathway can be assessed e.g. by measuring amount or composition of one or several polypeptides or metabolites belonging to the biological network and/or to the metabolic pathway from a biological sample taken from a subject. Risk to develop obesity is evaluated by comparing observed status and/or function of biological networks and or metabolic pathways of a subject to the status and/or function of biological networks and or metabolic pathways of healthy and obese subjects.

Another major application of the current invention is diagnosis of a molecular subtype of obesity in a subject. Molecular diagnosis methods and kits of this invention can be applied to a person being obese. In one preferred embodiment, molecular subtype of obesity in an individual is determined to provide information of the molecular etiology of obesity. When the molecular etiology is known, better diagnosis and prognosis of obesity can be made and efficient and safe therapy for treating obesity in an individual can be selected on the basis of this subtype information. Physicians may use the information on genetic risk factors with or without known clinical risk factors to convince particular patients to adjust their life style and manage obesity risk factors and select intensified preventive and curative interventions for them. In other embodiment, biomarker information obtained from methods and kits for determining molecular subtype of obesity in an individual is for monitoring the effectiveness of their treatment. In one embodiment, methods and kits for determining molecular subtype of obesity are used to select human subjects for clinical trials testing obesity foods. The kits provided for diagnosing a molecular subtype of obesity in an individual comprise wholly or in part protocol and reagents for detecting one or more biomarkers and interpretation software for data analysis and obesity molecular subtype assessment.

The diagnostic assays and kits of the invention may further comprise a step of combining non-genetic information with the biomarker data to make risk assessment, diagnosis or prognosis of obesity. Useful non-genetic information comprises age, gender, smoking status, physical activity, waist-to-hip circumference ratio (cm/cm), the subject family history of obesity, previously identified glucose intolerance, hypertriglyceridemia, low HDL cholesterol, HT and elevated BP. The detection method of the invention may also further comprise a step determining blood, serum or plasma glucose, total cholesterol, HDL cholesterol, LDL cholesterol, triglyceride, apolipoprotein B and AI, fibrinogen, ferritin, transferrin receptor, C-reactive protein and insulin concentration.

The score that predicts the probability of developing obesity may be calculated e.g. using a multivariate failure time model or a logistic regression equation. The results from the further steps of the method as described above render possible a step of calculating the probability of obesity using a logistic regression equation as follows. Probability of obesity=1/[1+e (−(−a+Σ(bi*Xi))], where e is Napier's constant, Xi are variables related to the obesity, bi are coefficients of these variables in the logistic function, and a is the constant term in the logistic function, and wherein a and bi are preferably determined in the population in which the method is to be used, and Xi are preferably selected among the variables that have been measured in the population in which the method is to be used. Preferable values for bᵢ are between −20 and 20; and for i between 0 (none) and 100,000. A negative coefficient bᵢ implies that the marker is risk-reducing and a positive that the marker is risk-increasing. Xi are binary variables that can have values or are coded as 0 (zero) or 1 (one) such as SNP markers. The model may additionally include any interaction (product) or terms of any variables Xi, e.g. biXi. An algorithm is developed for combining the information to yield a simple prediction of obesity as percentage of risk in one year, two years, five years, 10 years or 20 years. Alternative statistical models are failure-time models such as the Cox's proportional hazards' model, other iterative models and neural networking models.

Diagnostic test kits (e.g. reagent kits) of this invention comprise reagents, materials and protocols for assessing one or more biomarkers, and instructions and software for comparing the biomarker data from a subject to biomarker data from obese and non-obese people to make risk assessment, diagnosis or prognosis of obesity. Useful reagents and materials for kits comprise PCR primers, hybridization probes and primers as described herein (e.g., labeled probes or primers), allele-specific oligonucleotides, reagents for genotyping SNP markers, reagents for detection of labeled molecules, restriction enzymes (e.g., for RFLP analysis), DNA polymerases, RNA polymerases, DNA ligases, marker enzymes, antibodies which bind to polypeptides related to one or more obesity associated SNP markers disclosed in Tables 6 through 17, means for amplification and/or nucleic acid sequence analysis of nucleic acid fragments containing one or more obesity associated SNP markers set forth in Tables 6 through 17. In one embodiment, a kit for diagnosing susceptibility to obesity comprises primers and reagents for detecting the nucleotides present in one or more SNP markers selected from the Tables 6 through 17 of this invention in individual's nucleic acid.

Yet another application of the current invention is related to methods and test kits for monitoring the effectiveness of a treatment for obesity. The disclosed methods and kits comprise taking a tissue sample (e.g. peripheral blood sample or adipose tissue biopsy) from a subject before starting a treatment, taking one or more comparable samples from the same tissue of the subject during the therapy, assessing expression (e.g., relative or absolute expression) of one or more genes related to one or more obesity associated SNP markers set forth in Tables 6 through 17 of this invention in the collected samples of the subject and detecting differences in expression related to the treatment. Differences in expression can be assessed from mRNAs and/or polypeptides related to one or more obesity associated SNP markers set forth in Tables 6 through 17 of this invention and an alteration in the expression towards the expression observed in the same tissue in healthy non-obese individuals indicates the treatment is efficient. In a preferred embodiment the differences in expression related to a treatment are detected by assessing biological activities of one or more polypeptides related to one or more obesity associated SNP markers set forth in Tables 6 through 17 of this invention.

Based on the results disclosed below, the present invention is especially directed to a method for risk assessment, diagnosis or prognosis of obesity or type 2 diabetes (T2D) in a mammalian subject comprising:

a) providing a biological sample taken from the subject;

b) detecting one or more T2D and/or obesity associated genetic markers in said sample, wherein the genetic markers are related to SUCLA2 gene, and;

c) comparing the genetic marker data from the subject to genetic marker data from healthy and diseased people to make risk assessment, diagnosis or prognosis of obesity or T2D.

Accordingly, the invention is also directed to a test kit for risk assessment, diagnosis or prognosis of obesity or T2D comprising:

a) reagents, materials and protocols for assessing type and/or level of one or more T2D and/or obesity phenotype associated genetic markers in a biological sample, wherein the genetic markers are related to SUCLA2 gene, and;

b) instructions and software for comparing the genetic marker data from a subject to genetic marker data from healthy and diseased people to make risk assessment, diagnosis or prognosis of obesity or T2D.

### EXPERIMENTAL SECTION

### Example 1. Genotyping and Statistical Analyses of the GWS Data

Genomic DNA Isolation and Quality Testing

High molecular weight genomic and mitochondrial DNA was purified from frozen blood samples using QIAamp DNA Blood Midi kits (Qiagen). Concentration of purified DNA in each sample was measured using NanoDrop ND-1000 spectrophotometer (NanoDrop Technologies, Wilmington, Delaware USA) and aliquot was diluted to concentration 60 ng/ul. A sample was qualified if A260/A280 ratio was ≧1.7.

Genome-Wide Scanning Using Illumina's HumanHap550

The whole-genome genotyping of the DNA samples was performed by using Illumina's Sentrix HumanHap550 BeadChips and Infinium II genotyping assay. The HumanHap550 BeadChips contained over 550,000 SNP markers of which majority were tagSNP markers derived from the International HapMap Project. TagSNPs are loci that can serve as proxies for many other SNPs. The use of tagSNPs greatly improves the power of association studies as only a subset of loci needs to be genotyped while maintaining the same information and power as if one had genotyped a larger number of SNPs.

The Infinium II genotyping with the HumanHap550 BeadChips were performed according to the “Single-Sample BeadChip Manual process” described in detail in “Infinium™ II Assay System Manual” provided by Illumina (San Diego, Calif., USA). Briefly, 750 ng of genomic DNA from a sample was subjected to whole-genome amplification. The amplified DNA was fragmented, precipitated and resuspended to hybridization buffer. The resuspended sample was heat denatured and then applied to one Sentrix HumanHap550 BeadChip. After overnight hybridization mis- and non-hybridized DNA was washed away from the BeadChip and allele-specific single-base extension of the oligonucleotides on the BeadChip was performed in a Tecan GenePaint rack, using labeled deoxynucleotides and the captured DNA as a template. After staining of the extended DNA, the BeadChips were washed and scanned with the BeadArray Reader (Illumina) and genotypes from samples were called by using the BeadStudio software (Illumina).

Assessment of Diet

Intake of nutrients was assessed by 177-item food frequency questionnaire.

Initial SNP Selection for Statistical Analysis

Prior to the statistical analysis, SNP quality was assessed on the basis of three values: the call rate (CR), minor allele frequency (MAF), and Hardy-Weinberg equilibrium (H-W). The CR is the proportion of samples genotyped successfully. It does not take into account whether the genotypes are correct or not. The call rate was calculated as: CR=number of samples with successful genotype call/total number of samples. The MAF is the frequency of the allele that is less frequent in the study sample. MAF was calculated as: MAF=min(p,q), where p is frequency of the SNP allele ‘A’ and q is frequency of the SNP allele ‘B’; p=(number of samples with “AA”-genotype+0.5*number of samples with “AB”-genotype)/total number of samples with successful genotype call; q=1−p. SNPs that are homozygous (MAF=0) cannot be used in genetic analysis and were thus discarded. H-W equilibrium is tested for controls. The test is based on the standard Chi-square test of goodness of fit. The observed genotype distribution is compared with the expected genotype distribution under H-W equilibrium. For two alleles this distribution is p2, 2pq, and q2 for genotypes ‘AA’, ‘AB’ and ‘BB’, respectively. If the SNP is not in H-W equilibrium it can be due to genotyping error or some unknown population dynamics (e.g. random drift, selection).

Markers with CR>90%, MA>1%, and H-W equilibrium Chi-square test statistic<27.5 (the control group) were used in the statistical analysis. A total of 315,917 Illumina300K SNPs fulfilled the above criteria and 534,022 Illumina550K SNPs.

Single SNP Analysis BMI and WHR (Binary Traits)

In our study the obese cases (based on BMI) had BMI>=30 and at least 1 obese relative and the obese controls had BMI<=27 and no obese relatives. Based on these selection criteria there were 128 obese cases and 522 controls.

Obesity was also defined based on WHR (waist to hip circumference ratio). In this case the obese cases (based on WHR) had WHR>=0.92 (men) or WHR>=0.83 (women) and at least one obese relative. Obese controls (based on WHR) had WHR<=0.91 (men) and WHR<=0.82 (women) and no obese relatives. Based on these selection criteria there were 311 cases (105 men and 206 women) and 303 controls (92 men and 211 women). The analyses were done for both genders combined and separately for men and women.

Differences in allele distributions between cases and controls were screened for all SNPs. The screening was carried out using the standard Chi-square independence test with 1 df (allele distribution, 2×2 table). SNPs that gave a P-value less than 0.001 (Chi-square with 1 df of 10.23 or more) were considered statistically significant and reported in Tables 6 through 17. Odds ratio was calculated as ad/bc, where a is the number of minor alleles in cases, b is the number of major alleles in cases, c is the number of minor allele in controls, and d is the number of major alleles in controls. Minor allele was defined as the allele for a given SNP that had smaller frequency than the other allele in the control group.

Single SNP Analysis BMI and WHR (Continuous Traits)

10-based logarithm transformation was used for BMI values and samples with log(BMI)>1.6 were discarded from the analysis as outliers. Our data set included 1191 log(BMI) samples.

WHR values were first adjusted for gender, smoking, physical activity, alcohol g/week, and age. Samples having WHR residual>3 or WHR residual<−3 were excluded from the WHR analysis. Our data set included 1203 subjects with adjusted WHR values.

The data were analyzed using PLINK-program where the sample means of the two groups with different alleles were compared with the t-test.

It was invented that one can use the ratio of BMI and WHR to dietary energy intake as a measure of “energy intolerance” or “energy efficiency”. The same can be technically done by examining the interaction of BMI and WHR with energy intake.

### Example 2. SUCLA2 Gene Polymorphisms Modify the Association Between Energy Intake and Obesity

In our 550 k GWS data set, SNPs in the gene sucla2 were associated with the ratio of BMI and WHR to dietary energy intake and also modified the energy intake×BMI and energy intake×WHR interactions. The gene works in the Krebs cycle. The function and possibly activity of the gene can be assessed by measuring its metabolites in urine (see, e.g., prior art technologies disclosed in US 5,508,204 and Williams et al., 2005, J. Pharm. Biomed. Anal. 38(3):465-471). The invention concerns the diagnostic use of markers in the sucla2 gene, the sucla2 gene as target for obesity drugs and the use of sucla2 metabolites in monitoring energy efficiency and tolerance, energy consumption and physical activity. These markers can be either genetic, RNA, protein markers or metabolites of sucla2.

Over 550,000 gene-tagging SNP markers were typed in 1062 subjects from East Finland.

If we compare two persons with a same BMI:
high value in BMI/E means that a person with the same BMI gets (eats) less energy in her/his diet than the person with a low BMI/E
low value means that a person with the same BMI takes in more energy i.e. can eat more and still does not get any more obese

If we compare two persons with a same Energy intake:
high value in BMI/E means that the person has higher BMI than the person with a low BMI/E, both at the same energy intake

The person with a high BMI/E value tends to store the energy easier or at lower energy intake levels than a person with a low BMI/E. I.e. the lower the ratio, the larger is the proportion of energy used out of taken energy. A high BMI/E ratio denotes energy intolerance, i.e. BMI tends to rise easier or at a lower energy intake levels.

Mean values of BMI/E (Ratio of BMI to Energy Intake) in Subjects with Different RS12873870 Genotypes: AA and AG Versus GG Genetypes (GG vs Other)

TABLE 2
The distribution of BMI/E in all 1062 subjects
according to RS12873870 genotype.

| | | | Std. | Std. Error |
|---|---|---|---|---|
| GENOTYPE | N | Mean | Deviation | Mean |
| GG | 989 | 0.014777 | 0.005373 | 0.000170837 |
| AA or AG | 73 | 0.018016 | 0.00606 | 0.000709282 |

P-value for difference: 9.71E-07. Allele A carriers are energy intolerant, as compared with the GG homozygotes.

Difference in BMI/E Between Genders

TABLE 3
The distribution of BMI/E in all 1062 subjects in men and women.

| | | | Std. | Std. Error |
|---|---|---|---|---|
| GENDER | N | Mean | Deviation | Mean |
| MEN | 378 | 0.014171 | 0.005273 | 0.000271204 |
| WOMEN | 684 | 0.015457 | 0.005544 | 0.000211975 |

P-value: 2.43E-04.

Women are more energy intolerant (less energy tolerant) than men and the least energy tolerant are those women with either AA or AG genotype of RS 12873870.

Comparison of Different Collected and Measured Measurements Between RS12873870 A-and GG Genotypes

Thus, the RS12873870 genotype was also associated with many obesity-related traits such as hsCRP (C-reactive protein), height and dietary intakes of energy, starch, total sugars, fat, protein, insoluable fiber, and cholesterol. The individuals with the rare (mutant) allele, A, had lower intakes of all energy nutrients, but were more obese and had much higher serum CRP. It can be speculated that there was enhanced inflammatory response in them or metabolic changes in the liver and pancreatic carbohydrate metabolism, which manifested as elevated CRP.

A high CRP is associated with obesity and elevated leptin and elevated leptin to adiponectin ratio. This is consistent with a relationship between obesity, glucose homeostasis, and inflammation. Our invention also suggests that the RS 12873870 genotype may be a very early predictor of obesity, insipient insulin resistance, glucose intolerance and T2D. Individuals with defective SUCLA2 function could be prone for fat accumulation due to reduced functioning of the Krebs cycle.

TABLE 4
Statistical significance of associations
or differences in all 1062 subjects.

| ANOVA Table | F | Sig. |
|---|---|---|
| bmi_per_energy * GENOTYPE | 24.26882 | 9.71E−07 |
| whr_per_energy * GENOTYPE | 13.8493 | 0.000208 |
| Starch (g) * GENOTYPE | 13.55887 | 0.000243 |
| Total energy intake (kJ) * GENOTYPE | 12.59436 | 0.000404 |
| Fat (g)* GENOTYPE | 10.39617 | 0.001301 |
| Protein (g) * GENOTYPE | 10.1147 | 0.001513 |
| Serum High sensitivity CRP (mg/l) * | 9.989428 | 0.001614 |
| GENOTYPE | | |
| Insoluable fiber (g) * GENOTYPE | 9.628912 | 0.001966 |
| Total sugars (in food and added) (g) * | 5.046629 | 0.024878 |
| GENOTYPE | | |
| Height cm * GENOTYPE | 4.385863 | 0.036446 |
| Dietary cholesterol (mg) * GENOTYPE | 4.061926 | 0.044111 |

TABLE 5
The distribution of different traits in all 1062 subjects according to RS12873870 genotype.

| | | | | | | | Serum |
|---|---|---|---|---|---|---|---|
| | | | | | | | High |
| | | | | | | Body | sensitivity |
| | | BMI/ | WHR/ | Weight | Height | Mass | CRP |
| GENOTYPE | | energy | energy | kg | cm | Index | (mg/l) |
| GG | Mean | 0.014777 | 0.000485 | 74.6667 | 166.3077 | 26.93844 | 1.92975 |
| | N | 989 | 989 | 1120 | 1120 | 1120 | 1120 |
| | Std. | 0.005373 | 0.00016 | 14.51929 | 8.617389 | 4.544074 | 3.22724 |
| | Deviation | | | | | | |
| AA + AG | Mean | 0.018016 | 0.000558 | 74.34382 | 164.309 | 27.58928 | 3.06348 |
| | N | 73 | 72 | 89 | 89 | 89 | 89 |
| | Std. | 0.00606 | 0.00017 | 12.26919 | 9.259364 | 4.39697 | 3.61529 |
| | Deviation | | | | | | |
| Total | Mean | 0.015 | 0.000489 | 74.64293 | 166.1605 | 26.98635 | 2.01320 |
| | N | 1062 | 1061 | 1209 | 1209 | 1209 | 1209 |
| | Std. | 0.005481 | 0.000162 | 14.36145 | 8.677937 | 4.534823 | 3.26919 |
| | Deviation | | | | | | |

| | Total | | | | Total sugars | Water |
|---|---|---|---|---|---|---|
| | energy | | Protein | | (natural and | unsoluable |
| GENOTYPE | (kJ) | Fat (g) | (g) | Starch (g) | added) (g) | fiber (g) |
| GG | 8446.393 | 63.37012 | 92.81167 | 140.9142 | 114.2726 | 21.74235 |
| | 994 | 994 | 994 | 994 | 994 | 994 |
| | 3285.906 | 28.83443 | 36.03632 | 62.36105 | 59.337 | 10.62005 |
| AA + AG | 7066.014 | 52.35541 | 79.22973 | 113.7405 | 98.47568 | 17.84459 |
| | 74 | 74 | 74 | 74 | 74 | 74 |
| | 2299.894 | 20.67073 | 26.02831 | 43.26289 | 42.86433 | 7.256233 |
| Total | 8350.749 | 62.60693 | 91.8706 | 139.0314 | 113.1781 | 21.47228 |
| | 1068 | 1068 | 1068 | 1068 | 1068 | 1068 |
| | 3245.495 | 28.47501 | 35.59227 | 61.60282 | 58.468 | 10.46644 |

Information on RS12873870 Association with SUCLA2 and BMI Per Energy Intake

SUCLA2, GeneID:8803, mRNA NM_{—}003850.2, genomic reference NC_{—}000013.10, position

Chr 13 (13q12.2-q13.3),

Start: 47,414,792 by from pter

End: 47,473,463 by from pter

Size: 58,672 bases

Orientation: minus strand

Analysis results BMI per energy intake

Significant SNP:

| Marker | n | P | MAF | CR | Chr | pos | gene |
|---|---|---|---|---|---|---|---|
| RS12873870 | 1062 | 1.11E−06 | 0.037634 | 1 | 13 | 47443974 | SUCLA2 |

intron
Minor allele A // major allele G
Total of 4 intragenic SNPs are in Illumina 550k assay. RS12873870 is potentially obesity-associated, p for BMI = 0.003105

NCBI dbSNP for RS12873870
MAF_CEU: 0.067 (NOTE: within populations genotyped in HapMap project, this SNP is polymorphic only in Caucasian population)
Alleles: C/T forward; A/G reverse

LD in HapMap CEU population: RS12873870 is in D′=1 with a number of markers, but is in relatively low R² with all markers. The highest R²=0.38 with 9 SNPs that are intronic/flanking 5′/3′ to SUCLA2, and intronic/flanking 5′ to MED4 (gene ID: 29079). Thus, the observed association of RS12873870 indicates association of SUCLA2 gene.

LD Block Structure: SUCLA2 shares an LD block with the neighboring 5′ genes NUDT15 (Nucleoside diphosphate-linked moiety×motif 15) and MED4. RS12873870 is an ‘outlier’ in the LD block having very little linkage to other markers in the block.

LD in the Eastern Finnish Population: Similar to HapMap CEU population; significant R2=0.405 with rs9285165 (intergenic, p=0.06498 in BMI per energy)

Gender Distribution of RS12873870 Minor Allele (AA/AG) Frequency in the Eastern Finnish Population:

There is gender specificity in SUCLA2 RS 12873870 minor allele (A) inheritance. The minor allele A is more frequent in females than in males.

SUCLA2 Markers in Affymetrix 100K Mapping Assay:

100 K assay has two SNPs for SUCLA2 gene, intronic RS2182374 and a locus-region SNP RS7335797. Neither of these SNPs is in Illumina 500 k assay.

Information on 2 Gene:

The SUCLA2 gene encodes the beta-subunit of the ADP-forming succinyl-CoA synthetase (SCS-A; EC 6.2.1.5). SCS is a mitochondrial matrix enzyme that catalyzes the reversible synthesis of succinyl-CoA from succinate and CoA. The reverse reaction occurs in the Krebs cycle, while the forward reaction may produce succinyl-CoA for activation of ketone bodies and heme synthesis. GTP-specific (SCS-G; EC 6.2.1.4) and ATP-specific (SCS-A) isoforms of SCS catalyze GTP-dependent and ATP-dependent reactions, respectively. SCS is composed of an invariant alpha subunit and a beta subunit that determines the enzyme's nucleotide specificity.

Synonyms: EC 6.2.1.5; ATP-specific succinyl-CoA synthetase subunit beta; Succinyl-CoA synthetase, betaA chain; SCS-betaA ; Renal carcinoma antigen NY-REN-39

Entrez Gene: The protein encoded by this gene is an ATP-specific SCS beta subunit that dimerizes with the SCS alpha subunit to form SCS-A, an essential component of the tricarboxylic acid cycle. SCS-A hydrolyzes ATP to convert succinate to succinyl-CoA. Defects in this gene are a cause of myopathic mitochondrial DNA depletion syndrome. A pseudogene of this gene has been found on chromosome 6.

Map: This gene SUCLA2 maps on chromosome 13, at 13q12.2-q13.3 according to Entrez

Gene. In AceView, it covers 228.41 kb, from 47510103 to 47281697 (NCBI 36, March 2006), on the reverse strand.

AceView (shortened): RefSeq annotates one representative transcript (NM included in

AceView variant.c), but Homo sapiens cDNA sequences in GenBank, filtered against clone rearrangements, coaligned on the genome and clustered in a minimal non-redundant way by the manually supervised AceView program, support at least 17 spliced variants. Alternative mRNA expression and splicing: The gene contains 36 different gt-ag introns. Transcription produces 20 different mRNAs, 17 alternatively spliced variants and 3 unspliced forms. 659 by of this gene are antisense to spliced gene blaspey, 399 to NUDT15, raising the possibility of regulated alternate expression. Protein coding potential: 13 spliced and the unspliced mRNAs putatively encode good proteins, altogether 14 different isoforms (10 complete, 2 COOH complete, 2 partial).

Several transcripts of various sizes are coded for SUCLA2 gene thus suggesting existence of multiple protein variants.

SwissProt: Pathway: Carbohydrate metabolism; tricarboxylic acid cycle.

Protein length is 463 amino acids. It is a precursor protein; it contains a 52 amino acid long mitochondrial sorting sequence, and a 411 amino acids long Succinyl-CoA ligase [ADP-forming] subunit beta, mitochondrial sequence. Molecular weight: 50317 Da.

Tissue Specificity: Widely expressed. SUCLA2 is predominant in catabolic tissues, such as brain, heart, and skeletal muscle. Expression as well as the amount of the protein and enzymatic activity of SCS-A varies considerably between tissues in one species but also between species (Lambeth D O, Tews K N, Adkins S, Frohlich D, Milavetz B I. Expression of two succinyl-CoA synthetases with different nucleotide specificities in mammalian tissues. J Biol Chem. 2004 Aug. 27;279(35):36621-4.).

Posttranslational Modification: phosphoprotein (Rattus norvegicus): the alpha-subunit of succinyl-CoA synthetase undergoes autophosphorylation at a histidine residue. Coprovision of exogenous succinate and CoA results in pronounced dephosphorylation of the phosphorylated alpha-subunit of succinyl-CoA synthetase (source BRENDA database)

Pathways for SUCLA2

KEGG pathway: CS-Branched dibasic acid metabolism (00660)

KEGG pathway: Citrate cycle (TCA cycle) (00020)

KEGG pathway: Propanoate metabolism (00640)

KEGG pathway: Reductive carboxylate cycle (CO2 fixation) (00720)

Reactome Event: Pyruvate metabolism and TCA cycle (71406)

SUCLA2 Associated Phenotypes:

OMIM: Deficiency of SUCLA2 is associated with encephalomyopathy and mitochondrial DNA depletion.

Literature References for the Phenotype:

1. The mitochondrial DNA (mtDNA) depletion syndrome is a quantitative defect of mtDNA resulting from dysfunction of one of several nuclear-encoded factors responsible for maintenance of mitochondrial deoxyribonucleoside triphosphate (dNTP) pools or replication of mtDNA. Markedly decreased succinyl-CoA synthetase activity due to a deleterious mutation in SUCLA2, the gene encoding the beta subunit of the ADP-forming succinyl-CoA synthetase ligase, was found in muscle mitochondria of patients with encephalomyopathy and mtDNA depletion. Succinyl-CoA synthetase is invariably in a complex with mitochondrial nucleotide diphosphate kinase; hence, the authors propose that a defect in the last step of mitochondrial dNTP salvage is a novel cause of the mtDNA depletion syndrome (Elpeleg o, et al.,: Deficiency of the ADP-forming succinyl-CoA synthase activity is associated with encephalomyopathy and mitochondrial DNA depletion. Am J Hum Genet. 2005 June;76(6):1081-6. Epub 2005 Apr. 22.).

2. “The hallmark of the condition, elevated methylmalonic acid, can be explained by an accumulation of the substrate of the enzyme, succinyl-CoA, which in turn leads to elevated methylmalonic acid, because the conversion of methylmalonyl-CoA to succinyl-CoA is inhibited.” (Ostergaard E, Hansen F J, Sorensen N, Duno M, Vissing J, Larsen P L, Faeroe O, Thorgrimsson S, Wibrand F, Christensen E, Schwartz M. Mitochondrial encephalomyopathy with elevated methylmalonic acid is caused by SUCLA2 mutations. Brain. 2007 March;130(Pt 3):853-61.)

3. Succinate-CoA ligase catalyses the reversible conversion of succinyl-CoA and ADP or GDP to succinate and ATP or GTP. It is a mitochondrial matrix enzyme and at least the ADP-forming enzyme is part of the Krebs cycle. The substrate specificity is determined by the beta subunit of succinate-CoA ligase, which is encoded by either SUCLA2 or SUCLG2. In patients with severe hypotonia, deafness and Leigh-like syndrome, mutations have been found in SUCLA2. Mutations have also been reported in SUCLG1, which encodes the alpha subunit found in both enzymes, in patients with severe infantile acidosis and lactic aciduria. Elevated methylmalonate and methylcitrate and severe mtDNA depletion were found in both disorders. The mtDNA depletion may be explained by the interaction of succinate-CoA ligase with nucleoside diphosphate kinase, which is involved in mitochondrial nucleotide metabolism (Ostergaard E. Disorders caused by deficiency of succinate-CoA ligase. J Inherit Metab Dis. 2008 Apr. 4.).

What is encephalomyopathy?

Mitochondrial encephalomyopathy-aminoacidopathy: A very rare syndrome characterized mainly by muscle and brain disease and an amino acid disorder. Medical symptoms include: Developmental delay, Neurological problems, Deafness, Exercise intolerance, Lactic acidosis, Increased level of amino acids in plasma, Muscle wasting, Reduced reflexes, Ataxia, and Poorly muscled build.

Muscle Tissue in Subjects with SUCLA2 Deficiency: Histology of muscle tissue showed a very consistent and characteristic pattern in all seven patients from whom a muscle biopsy was available. The findings included (i) increased variability of fibre diameter with scattered hypertrophic, spherical fibres with an increased mitochondrial content, (ii) a marked type I fibre predominance (>95%) and (iii) extensive intracellular fat accumulation in type I fibres (Ostergaard E, Hansen F J, Sorensen N, Duno M, Vissing J, Larsen P L, Faeroe O, Thorgrimsson S, Wibrand F, Christensen E, Schwartz M. Mitochondrial encephalomyopathy with elevated methylmalonic acid is caused by SUCLA2 mutations. Brain. 2007 March;130(Pt 3):853-61.). We propose as part of this invention that intracellular fat accumulation may be due to muscular atrophy that is caused by decreased mtDNA in SUCLA2 deficiency.

Role of SCS-A in TCA Cycle:

SCS-A plays a significant role in Citric acid cycle. Entrez Gene and other databases present the function of SUCLA2 in hydrolyzing ATP to convert succinate to succinyl-CoA. However, it appears that SCS-A complex in fact catalyzes the reverse reaction in the citric acid cycle. Succinyl-CoA+ADP→succinate+CoA+ATP (D. O. Lambeth, K. N. Tews, S. Adkins, D. Frohlich, and B. I. Milavetz: Expression of Two Succinyl-CoA Synthetases with Different Nucleotide Specificities in Mammalian Tissues. J. Biol. Chem., Aug. 27, 2004; 279(35): 36621-36624.) SCS-A is not a rate limiting enzyme in Krebs cycle. Its activity is regulated by the amount of succinyl-CoA. In KEGG TCA-pathway, enzyme Succinyl-CoA hydrolase (EC 3.1.2.3) is presented as functionally similar enzyme for conversion of Succinyl-CoA to succinate. This enzyme, however, has been described only in organisms in lower taxonomy, and thus cannot be considered as relevant substitute for SCS-A/SCS-G enzymes.

Invention Concerning the Role of SUCLA2 in Energy Intolerance:

Both SCS-A and SCS-G are localized in beta cell mitochondria, and it has been proposed that GTP generated by the activation of succinylCoA synthetase could promote key functional roles in the mitochondrial metabolism leading to insulin secretion (Kowluru A. Diabetologia. 2001 January;44(1):89-94. Adenine and guanine nucleotide-specific succinyl-CoA synthetases in the clonal beta-cell mitochondria: implications in the beta-cell high-energy phosphate metabolism in relation to physiological insulin secretion. PMID: 11206416).

Knockdown of SCS-A (by si-RNA) in rat INS-1832/13 insulinoma cells and in cultured rat islets increases glucose-stimulated insulin secretion (GSIS) by two-fold, whereas suppression of GTP-specific SCS (SCS-G) reduces GSIS by 50%. Increasing the rate of GTP synthesis by reducing the expression of SCS-ATP results in increased oxygen consumption and cytosolic calcium with a concomitant increase in insulin secretion, which is unassociated with an increase in the ATP/ADP ratio or NAD(P)H. Conversely, if GTP synthesis is decreased by silencing SCS-GTP, then oxygen consumption, ATP synthesis, and NAD(P)H levels increase while cytosolic calcium does not, leading to impaired GSIS. Taken together, these data suggest that TCA-cycle-generated mtGTP regulates insulin secretion by increasing cytosolic calcium (Kibbey R G, Pongratz R L, Romanelli A J, Wollheim C B, Cline G W, Shulman G I. Mitochondrial GTP regulates glucose-stimulated insulin secretion. Cell Metab. 2007 April;5(4):253-64.).

Therefore, it is plausible that alterations in SUCLA2 function would influence glucose-induced insulin secretion. In particular, decreased function of SCS-A could provide more availability of succinyl-CoA for SCS-G to promote GTP production and subsequently glucose stimulated insulin secretion. Hypothetically, subjects RS12873870 minor allele A genotype could have such alterations in their insulin secretion that would promote energy intolerance.

In Krebs cycle, SCS-A catalyzes the synthesis of succinate+CoA+ATP from succinyl-CoA and ADP. Thus, increased expression or activity of SCS-A could lead to accumulation of succinate in Krebs cycle, which is substrate for fumarate production. Obesity-associated gene FTO that encodes for a 2-Oxoglutarate-dependent nucleic acid demethylase is an enzyme that is inhibited by Krebs cycle intermediates, in particular by fumarate, but also by succinate (Gerken T et al., The obesity-associated FTO gene encodes a 2-oxoglutarate-dependent nucleic acid demethylase., Science. 2007 Nov 30;318(5855):1469-72.). Modulation of FTO activity has been suggested in disease states with elevated fumarate/succinate levels. →Thus, it is possible that a single point mutation affecting expression or activity of SUCLA2 gene may have a function that could indirectly relate with function of FTO that could result in energy intolerance. Increased production of succinate and fumarate, in particular, could inhibit FTO function. In animal models, FTO has been shown to be regulated by feeding and starvation. With this aspect, it is of interest that subjects with SUCLA2 RS12873870 minor allele A genotype seem to eat less and yet maintain same BMI as those with the major allele genotype.

Thus, minor allele A might relate to lower threshold for satiety, or increased tendency to gain weight which would be counteracted by intentional lower calorie consumption.

Interestingly, increased fumarate has been shown to induce adipogenesis in vitro. S-(2-succinyl)cysteine (2SC), the product of chemical modification of proteins by the Krebs cycle intermediate, fumarate, is significantly increased during maturation of 3T3-L1 fibroblasts to adipocytes (Nagai R, Brock J W, Blatnik M, Baatz J E, Bethard J, Walla M D, Thorpe S R, Baynes J W, Frizzell N. Succination of protein thiols during adipocyte maturation: a biomarker of mitochondrial stress. J Biol Chem. 2007 Nov 23;282(47):34219-28.).

In contrast, decreased expression or activity of SCS-A could lead to accumulation of succinyl-CoA in Krebs cycle. As succinyl-CoA functions as feedback inhibitor of Krebs cycle by inhibiting citrate synthase (Lalloue, Bryla and Williamson: Feedback inhibitions in the control of citric acid cycle activity in rat heart mitochondria. JBC, 1972), it is possible that reduced SCS-A function leads to reduced overall energy production in Krebs cycle. Concomitantly, Krebs cycle intermediates forward from succinate would be below normal levels as a result of decreased SCS-A activity. In combination, these two pathways when converging could result in elevated levels of mitochondrial acetyl-CoA.

Increased mitochondrial levels of acetyl-CoA could result in transportation of acetyl-CoA to cytosol via carnitine acetylcarnitine carrier complex. Cytosolic elevated levels of acetyl-CoA can result in increased conversion acetyl-CoA to Malonyl-CoA by the action of acetyl-CoA carboxylase (ACC). Malonyl-CoA is a potent inhibitor of CPT I (carnitine palmitolyltransferase I), and this inhibition could result in decreased mitochondrial fatty acid oxidation. Decreased fatty acid oxidation, in turn, results in abnormal fatty acid metabolism and storage. In this model, decreased overall activity of the citric acid cycle would yield elevated levels of fat, and subjects with defective SUCLA2 function would be prone for fat accumulation due to reduced functioning of the Krebs cycle. Furthermore, it has been proposed that malonyl-CoA serves as an intermediary in a signaling circuit that regulates feeding behavior (Dowell P., Hu Z., Lane MD. Annu. Rev. Biochem. 74, 515-534, 2005). Interestingly, in our BMI/Energy Intake—dataset, subjects with minor allele hetero/homozygotia in RS12873870 were not significantly different in BMI from control group. They however eat less and yet maintain normal BMI. This could potentially be explained by more efficient intake of energy from the food that might accompany with lower threshold for feeling satiety.

Potential Sites for Modulation of SCS-A

Interacting Partners of Succinyl-CoA Synthase (SCS):
Nucleoside diphosphate kinase (NDPK; alias mNDPK; NDPK-D, encoded by gene NME4). This association has been proposed to enable intramitochondrial generation of GTP which (unlike ATP) cannot be transported into mitochondria via classical nucleotide translocase. (Kowluru A, Tannous M, Chen H Q. Localization and characterization of the mitochondrial isoform of the nucleoside diphosphate kinase in the pancreatic beta cell: evidence for its complexation with mitochondrial succinyl-CoA synthetase. Arch Biochem Biophys. 2002 Feb. 15;398(2):160-9. PMID: 11831846). This complex formation has also been proposed to underlie the mitochondrial DNA defect in SUCLA2 gene phenotype (Ostergaard E. Disorders caused by deficiency of succinate-CoA ligase. J Inherit Metab Dis. 2008 Apr. 4.) NDPK is responsible for intracellular di-and triphosphonucleoside homeostasis, plays multifaceted role in cellular energetic, signaling, proliferation, differentiation, and tumor invasion. NDPK-D localizes in inner mitochondrial membrane and is suggested to function for mitochondrial membrane lipid transfer in liposomes that mimic mitochondrial membrane contents (Epand R F, Schlattner U, Wallimann T, Lacombe M L, Epand R M. Novel lipid transfer property of two mitochondrial proteins that bridge the inner and outer membranes. Biophys J. 2007 Jan. 1;92(1):126-37.). NDPK-D has also been recently shown to bind with high affinity to cardiolipin, and to couple with mitochondrial oxidative respiration (Tokarska-Schlattner M, Boissan M, Munier A, Borot C, Mailleau C, Speer O, Schlattner U, Lacombe M L. The nucleoside diphosphate kinase D (NM23-H4) binds the inner mitochondrial membrane with high affinity to cardiolipin and couples nucleotide transfer with respiration. J Biol Chem. 2008 Jul 17. [Epub ahead of print]).
TRIM28 interacts with the SUCLA2 gene. PMID 17542650. An interaction between TRIM28 and the SUCLA2 gene was demonstrated by ChIP-on-chip assay.TRIM28: Tripartite motif-containing 28. ID: 10155. GO Terms Molecular Function.transcription factor activity GO:3700.transcription corepressor activity GO:3714.protein binding GO:5515.zinc ion binding GO:8270.sequence-specific DNA binding GO:43565.metal ion binding GO:46872.electron transporter activity GO:5489 Cellular Component. intracellular GO:5622.nucleus GO:5634 Biological Process.epithelial to mesenchymal transition GO:1837.transcription GO:6350.regulation of transcription from RNA polymerase II promoter GO:6357.positive regulation of gene-specific transcription GO:43193.electron transport GO:6118
Pol II interacts with the SUCLA2 promoter. An interaction between Pol II (RNA polymerase II) and SUCLA2 promoter was demonstrated by chromatin immunoprecipitation and genomic microarray hybridization (chIp-CHIP). PMID 12808131.
E2F1 interacts with the SUCLA2 promoter. PMID 12808131. E2F transcription factor 1; retinoblastoma-associated protein 1; pRB-binding protein 3. E2F1 (RBP3) is a member of the E2F transcription factor family. E2F1 displays preferential binding to retinoblastoma protein pRB in a cell-cycle dependent manner, and is involved in cell proliferation and p53-dependent/independent apoptosis. NCBI Entrez 1869.
TAFII250 interacts with the SUCLA2 promoter. PMID 12808131. TAF1 RNA polymerase II, TATA box binding protein (TBP)-associated factor. Note that the listed GeneID refers to multiple variants encoded by the same gene. The precise molecular variant involved in this interaction is not specified. NCBI Entrez Gene Id 6872.
HNF4-alpha interacts with the SUCLA2 promoter region. PMID 14988562. Hepatocyte nuclear factor 4-alpha; transcription factor 14; hepatic nuclear factor. Mutations in this gene have been associated with monogenic autosomal dominant non-insulin-dependent diabetes mellitus type I. Three transcript variants encode three isoforms. This protein represents variant 2 and isoform b. NCBI ID: 3172. DNA binding GO:3677.transcription factor activity GO:3700.RNA polymerase II transcription factor activity GO:3702.steroid hormone receptor activity GO:3707.receptor activity GO:4872.ligand-dependent nuclear receptor activity GO:4879.steroid binding GO:5496NOTE: this is a well known molecule in diabetes mellitus.
ALAS2 interacts with SUCLA2 as identified by two hybrid. This is an elemental interaction record from MIPS. PMID 10727444. The first and the rate-limiting enzyme of heme biosynthesis is delta-aminolevulinate synthase (ALAS), which is localized in mitochondria. 5-aminolevulinic acid synthase, erythroid-specific, mitochondrial precursor. NCBI ID: 28588. ALAS2 interacts with SUCLA2 as identified by coimmunoprecipitation. This is an elemental interaction record from MIPS.
In other species, e.g. yeast, bacteria and fruit fly, also other interacting molecules have been described. However, the only small molecules are CoA, Mg2+, and ADP. Other molecule types belong to proteins, genes and DNA.

Hormone-sensitive lipase (HSL), a key enzyme in fatty acid mobilization in adipocytes knock-out mice showed increased expression in transcriptome analysis of soleus muscle of HSL-null mice of succinyl-CoA synthetase, (1.25 and 1.30) (Hansson O, Donsmark M, Ling C, Nevsten P, Danfelter M, Andersen J L, Galbo H, Holm C. Transcriptome and proteome analysis of soleus muscle of hormone-sensitive lipase-null mice. J Lipid Res. 2005 December;46(12):2614-23.). HSL is encoded in humans by the LIPE (HSL, GeneID: 3991, mRNA NM_{—}005357; genomic reference NC_{—}000019.9) gene. HSL is thus an activator of SCS-A, and recombinant HSL or analogs of HSL can be used as SCS-A agonists and to boost the Krebs cycle. In the Krebs cycle, SCS-A catalyzes the synthesis of succinate+CoA+ATP from succinyl-CoA and ADP. Thus, increased expression or activity of SCS-A could lead to accumulation of succinate in Krebs cycle, which is substrate for fumarate production. HSL may be activated by two mechanisms:
In the first, it is phosphorylated by perilipin A, causing it to move to the surface of the lipid droplet, where it may begin hydrolyzing the lipid droplet. Perilipin A is encoded in humans by the PLIN1 gene (GeneID: 5346, mRNA NM_{—}002666.4; genomic reference NC_{—}000015.9).
Alternately, it may be activated by a cAMP-dependent protein kinase, encoded in humans by the PRKACA gene (GeneID: 5566, mRNA NM_{—}002730.3; genomic reference NC_{—}000019.9). This pathway is significantly less effective than the first, which is necessary to lipid mobilization in response to cyclic AMP, which itself is provided by beta adrenergic stimulation of the glucagon receptor.

Thus, also recombinant forms or analogs of perilipin A or cAMP-dependent protein kinase may be used as agonists of SCS-A and to boost the Krebs cycle. Any biomarker or metabolite of the interacting proteins or activators can be used as biomarkers of sucla2.

### CONCLUSIONS

1. Marker RS12873870 supports association of SUCLA2 gene in BMI/energy data set.

2. Association of RS12873870 could relate to differential function or expression of SUCLA2 protein. Several transcripts have been described that in theory could have tissue specific roles. In addition, tissue specificity of SUCLA2 mRNA and protein has been described in humans.

3. SUCLA2 encodes for the beta subunit of ATP-specific succinyl-CoA ligase (SCS) that provides a part of the required ATP for citric acid cycle.

4. SCS has been shown to affect glucose stimulated insulin secretion in vitro.

5. Subjects with RS12873870 minor allele appear as energy intolerant. Minor allele subjects are not significantly different from the major allele genotype subjects by BMI, but they consume less energy for maintaining BMI. Distribution of muscle/fat ratio in the subjects under study is not known; it is possible that although BMI is not different, muscle/fat—ratio could be affected. CRP levels are higher in subjects with RS 12873870 minor allele genotype.

6. Potential sites for manipulation: transportation of citric acid cycle intermediates; modulation of SCS-A or SCS-G activity; modulation of methylmalonyl-CoA levels

### Example 3: Interactions Between SNPs, Intake of Energy Nutrients and Obesity (BMI or WHR) i.e. How SNPs Modify the Effect of Intakes of Energy, Fat and Carbohydrates on BMI and WHR

Linear Regression Between a Trait and a SNP

Subjects were from the Jukka T. Salonen's population study collected from the East-Finland founder population. Effect of the SNP variation were tested based on a simple linear regression where a dummy variable is a dose of the minor allele e.g. if the minor allele is A and the major allele is G then GG=0, AG=1, and AA=2. All calculations were based on PLINK-statistical package (http://pngu.mgh.harvard.edu/purcell/plink/) implemented in the BCISNPmax environment (Biocomputing platforms Ltd).

Following results, quality measurements and annotation information are presented:

BETA: Regression coefficient

R2: Regression r-squared

P: Wald test asymptotic p-value

HWE: Hardy-Weinberg equilibrium calculated for hypertension controls

MAF: minor allele frequency

CR: call rate

CHR: chromosome

POSITION: chromosomal position

GENE: gene if the SNP is intragenic

GENE_ID: gene ID

CLASS: classification of the intragenic SNP

Interaction between food intake and SNP

Each SNP was analyzed separately. The data were split into two subsets based on the SNP genotype: the first set included samples with minor allele of the SNP present and the other subset included samples that were homozygous for wild (major) allele. The following information was obtained for each SNP and subset:

B=regression coefficient

SE=standard error of the coefficient

t=t-test statistic for B=0 vs B≠0

P=P-value of the test statistic

The results of the two subsets were compared with the following statistic, that has a standard normal distribution:

$$z = \frac{{B}_{1} - {B}_{2}}{\sqrt{{SE}_{1}^{2} + {SE}_{2}^{2}}}$$

where the subscripts correspond to different subsets within a particular SNP. Explanations for the other abbreviations in the tables are following:

P-value P-value corresponding to z-value

HW Hardy-Weinberg equilibrium

MAF minor allele frequency

CR call rate

CHR chromosome

Position chromosomal position in by

Gene gene if the SNP is intragenic

GeneID corresponding gene ID

Class indicating if the intragenic SNP is intronic etc.

BMI per Energy Intake×SNP Interaction (“Energy Intolerance”)

Regression model (ln(BMI)=mu+energy+e, where energy intake in food) within different genotype groups. The model was separately used for samples with minor allele present and samples that are homozygous for the major allele.

SUMMARY: The closest gene of the significant SNP on chromosome is (KLF4) Kruppel-like factor 4 (gut), Gene ID:9314; mRNA NM_{—}004235.4, genomic reference NC_{—}000009.11.

TABLE 6
Continuous variable: ln(BMI) adjusted for age, HT-status, average weekly exercise.

| SNP | P | HW | MAF | CR | CHR | Position | Gene | GeneID | class |
|---|---|---|---|---|---|---|---|---|---|
| RS11792803 | 1.67E−08 | 0.224966 | 0.079385 | 0.995037 | 9 | 109563610 | | | |

| SNP | B1 | SE1 | P1 | n1 | B2 | SE2 | P2 | n2 | z | P |
|---|---|---|---|---|---|---|---|---|---|---|
| RS11792803 | −0.00012 | 2.56E−05 | 4.06E−06 | 153 | 3.19E−05 | 9.58E−06 | 0.00091 | 896 | −5.6439 | 1.67E−08 |

This SNP and associated biomarkers can be used for nutrigenetic diagnostics for the selection of individuals for low-energy food products.

BMI Per Fat Intake×SNP Interaction (“Fat Intolerance”)

SUMMARY: The closest gene of the significant SNP on chromosome is (KLF4) Kruppel-like factor 4 (gut).

TABLE 7
Continuous variable: ln(BMI), adjusted for age, HT-status, average weekly exercise.

| SNP | P | HW | MAF | CR | CHR | Position | Gene | GeneID | class |
|---|---|---|---|---|---|---|---|---|---|
| RS11792803 | 3.56E−08 | 0.224966 | 0.079385 | 0.995037 | 9 | 109563610 | | | |
| RS2046380 | 4.65E−07 | 0.108188 | 0.034326 | 1 | 3 | 178299225 | TBL1XR1 | 79718 | intron |
| RS2142100 | 5.38E−07 | 0.095324 | 0.011993 | 1 | 21 | 35806214 | | | |
| RS2834947 | 5.38E−07 | 0.082899 | 0.01158 | 1 | 21 | 35798818 | | | |
| RS11626428 | 6.26E−07 | 0.569168 | 0.147181 | 0.997519 | 14 | 94960193 | C14orf49 | 161176 | intron |
| RS6936924 | 7.76E−07 | 0.082899 | 0.011589 | 0.999173 | 6 | 34673137 | C6orf106 | 64771 | intron |

| SNP | B1 | SE1 | P1 | n1 | B2 | SE2 | P2 | n2 | z | P |
|---|---|---|---|---|---|---|---|---|---|---|
| RS11792803 | −0.01413 | 0.003042 | 7.30E−06 | 153 | 0.003664 | 0.00107977 | 0.00072 | 897 | −5.512 | 3.56E−08 |
| RS2046380 | 0.01678 | 0.003055 | 5.48E−07 | 73 | 0.000464 | 0.00107089 | 0.664928 | 983 | 5.040492 | 4.65E−07 |
| RS2142100 | 0.025645 | 0.004718 | 1.59E−05 | 23 | 0.001432 | 0.0010364 | 0.167227 | 1033 | 5.012513 | 5.38E−07 |
| RS2834947 | 0.025645 | 0.004718 | 1.59E−05 | 23 | 0.001432 | 0.0010364 | 0.167227 | 1033 | 5.012513 | 5.38E−07 |
| RS11626428 | 0.010674 | 0.002064 | 4.28E−07 | 294 | −0.00112 | 0.00116057 | 0.332994 | 759 | 4.983372 | 6.26E−07 |
| RS6936924 | 0.038005 | 0.007286 | 4.91E−05 | 19 | 0.001645 | 0.00103086 | 0.110955 | 1036 | 4.94154 | 7.76E−07 |

The SNPs and associated markers can be used for nutrigenetic diagnostics for the selection of individuals for low-fat food products.

BMI Per Carbohydrate Intake×SNP Interaction (“Carbohydrate Intolerance”)

SUMMARY: The closest gene of the significant SNP on chromosome is (KLF4) Kruppel-like factor 4 (gut).

TABLE 8
Continuous variable: ln(BMI) adjusted for age, HT-status, average weekly exercise.

| SNP | P-value | HW | MAF | CR | CHR | Position | Gene | GeneID | class |
|---|---|---|---|---|---|---|---|---|---|
| RS11792803 | 4.4441E−07 | 0.224966 | 0.079385 | 0.995037 | 9 | 109563610 | | | |
| RS2841959 | 1.9301E−06 | 0.814549 | 0.497874 | 0.972705 | 1 | 161324818 | | | |
| RS10906283 | 3.2046E−06 | 2.529734 | 0.086435 | 1 | 10 | 13101451 | | | |
| RS17491334 | 3.7129E−06 | 0.393652 | 0.102151 | 1 | 12 | 5974105 | VWF | 7450 | intron |
| RS16884072 | 5.0772E−06 | 0.127074 | 0.208023 | 1 | 6 | 20763482 | CDKAL1 | 54901 | intron |
| RS736425 | 5.0772E−06 | 0.127074 | 0.208023 | 1 | 6 | 20772291 | CDKAL1 | 54901 | intron |
| RS6492437 | 5.1895E−06 | 0.120972 | 0.39234 | 0.971878 | 13 | 89009740 | | | |
| RS4366776 | 5.8159E−06 | 4.341979 | 0.494157 | 0.990902 | 17 | 216763 | | | |
| RS10484632 | 6.589E−06 | 0.007833 | 0.216239 | 0.998346 | 6 | 20755639 | CDKAL1 | 54901 | intron |
| RS13194407 | 6.6385E−06 | 0.002442 | 0.196443 | 1 | 6 | 20738932 | CDKAL1 | 54901 | intron |
| RS2847666 | 7.5669E−06 | 0.117937 | 0.287375 | 0.995864 | 11 | 59616152 | MS4A2 | 2206 | intron |
| RS12684481 | 7.6028E−06 | 0.008928 | 0.0625 | 0.999173 | 9 | 109560355 | | | |
| RS13088837 | 8.2331E−06 | 0.076821 | 0.290323 | 1 | 3 | 63434165 | SYNPR | 132204 | intron |
| RS2191187 | 8.3962E−06 | 0.162998 | 0.206954 | 0.999173 | 12 | 125347217 | | | |
| RS8022938 | 9.5814E−06 | 7.494477 | 0.020281 | 0.999173 | 14 | 66925080 | PLEK2 | 26499 | intron |
| RS2841981 | 9.8281E−06 | 0.019897 | 0.487572 | 0.998346 | 1 | 161352134 | | | |

| SNP | B1 | SE1 | P1 | n1 | B2 | SE2 | P2 | n2 | z | P-value |
|---|---|---|---|---|---|---|---|---|---|---|
| RS11792803 | −0.00350834 | 0.00078 | 1.36E−05 | 153 | 0.000704 | 0.00029522 | 0.01728206 | 897 | −5.0492 | 4.44E−07 |
| RS2841959 | −0.00087331 | 0.000346 | 0.011801 | 760 | 0.001892 | 0.00046655 | 6.60E−05 | 264 | −4.76086 | 1.93E−06 |
| RS10906283 | −0.00297506 | 0.000721 | 5.83E−05 | 167 | 0.00066 | 0.00029851 | 0.02726645 | 889 | −4.65748 | 3.2E−06 |
| RS17491334 | −0.0024206 | 0.000633 | 0.000178 | 195 | 0.000833 | 0.00030537 | 0.00653528 | 861 | −4.62706 | 3.71E−06 |
| RS16884072 | −0.00178514 | 0.000528 | 0.000796 | 393 | 0.001033 | 0.00032059 | 0.00133622 | 663 | −4.56178 | 5.08E−06 |
| RS736425 | −0.00178514 | 0.000528 | 0.000796 | 393 | 0.001033 | 0.00032059 | 0.00133622 | 663 | −4.56178 | 5.08E−06 |
| RS6492437 | −0.00088902 | 0.000374 | 0.017631 | 653 | 0.00165 | 0.00041322 | 7.89E−05 | 370 | −4.55718 | 5.19E−06 |
| RS4366776 | 0.00078379 | 0.000314 | 0.012626 | 776 | −0.00235 | 0.00061536 | 0.00016942 | 271 | 4.533176 | 5.82E−06 |
| RS10484632 | −0.00174339 | 0.000522 | 0.000915 | 403 | 0.001023 | 0.00032304 | 0.0016164 | 651 | −4.50675 | 6.59E−06 |
| RS13194407 | −0.00184241 | 0.000543 | 0.000767 | 375 | 0.000993 | 0.00031776 | 0.0018625 | 681 | −4.50516 | 6.64E−06 |
| RS2847666 | −0.00118633 | 0.000436 | 0.00677 | 516 | 0.001318 | 0.00035013 | 0.00018458 | 537 | −4.47728 | 7.57E−06 |
| RS12684481 | −0.0033417 | 0.00083 | 9.77E−05 | 124 | 0.000597 | 0.00029263 | 0.0416818 | 931 | −4.47626 | 7.6E−06 |
| RS13088837 | −0.00104076 | 0.000389 | 0.007652 | 517 | 0.001413 | 0.00038955 | 0.00031317 | 539 | −4.45922 | 8.23E−06 |
| RS2191187 | −0.00149391 | 0.00047 | 0.001606 | 380 | 0.00109 | 0.00033978 | 0.001397 | 676 | −4.45501 | 8.4E−06 |
| RS8022938 | −0.00710126 | 0.001675 | 0.000124 | 41 | 0.000416 | 0.00027962 | 0.13747922 | 1014 | −4.42659 | 9.58E−06 |
| RS2841981 | −0.0007644 | 0.000346 | 0.027593 | 777 | 0.001783 | 0.00046058 | 0.0001348 | 277 | −4.4211 | 9.83E−06 |

The SNPs and associated markers can be used for nutrigenetic diagnostics for the selection of individuals for low-carbohydrate food products.

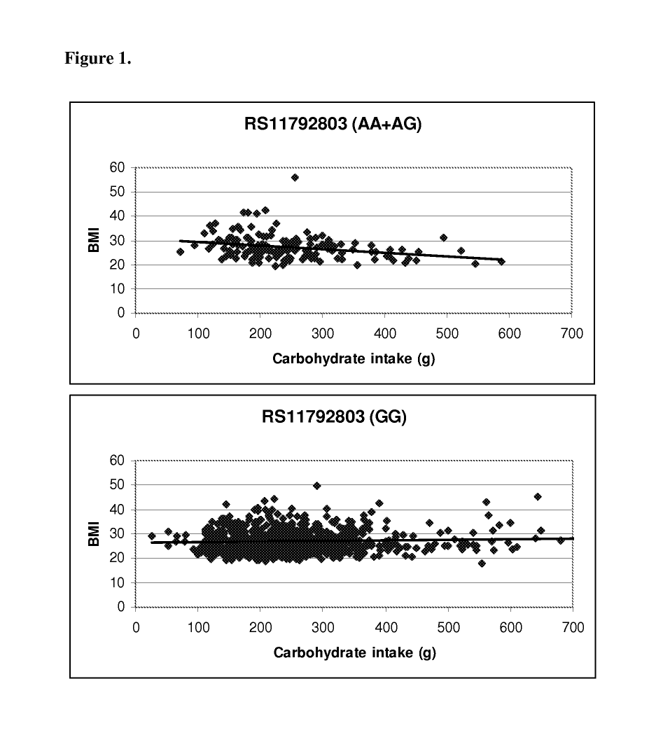

FIG. 1 shows linear regression between carbohydrate intake and BMI in genotypes of RS11792803.

Among the minor allele (A) carriers (upper panel), the higher the carbohydrate intake, the leaner the person. In this genotype group the individuals are more susceptible to fat than carbohydrates in gaining weight. In the majority of people (lower panel), there is a weak but significant association between carbohydrate intake and BMI. The gene in which several markers modify the effect of carbohydrate intake on BMI, is CDKAL1, a known type 2 diabetes gene. On the basis of this information, a nutrigenetic test could be constructed that would separate individuals who are likely to gain weight because of high carbohydrate intake. This can lead to nutrigenetic diagnostics for the selection of individuals for low-carbohydrate food products.

BMI Per Glycemic Load, Cumulative per Day×SNP Interaction (“Carbohydrate Intolerance”)

Calculation and Distributions of Carbohydrates, Glycemic Load, and Glycemic Index

The dietary glycemic load of each food was calculated by multiplying the carbohydrate content of one serving by the glycemic index. For example, the glycemic load of one serving of cooked potatoes was determined to be 38 because the carbohydrate content of one serving of potatoes is 37 g and the glycemic index of potatoes (with white bread as the reference) is 102% (ie, 1.02×37=38). We then multiplied this dietary glycemic load score by the frequency of consumption (1 time/d=1, 2-3 times/d=2.5, etc) and summed the products over all food items to produce the dietary glycemic load. The dietary glycemic load thus represents the quality and quantity of carbohydrates, and each unit of dietary glycemic load is the equivalent of 1 g carbohydrate from white bread.

Additionally, the overall dietary glycemic index—a variable representing the overall quality of carbohydrate intake for each participant—was created by dividing the dietary glycemic load by the total amount of carbohydrate consumed. Representation of the dietary glycemic load per unit of carbohydrate allowed for this measure to essentially match the carbohydrate content gram by gram and thus reflects the overall quality of the carbohydrate in the entire diet.

SUMMARY: The significant finding is an intronic SNP in MS4A2 gene (membrane-spanning 4-domains, subfamily A, member 2 (Fc fragment of IgE, high affinity I, receptor for; beta polypeptide)).

TABLE 9
Results from the glycemic load × SNP - interaction for BMI. Continuous variable: ln(BMI)
adjusted for: Age, HT-status, average weekly exercise.

| SNP | P-value | HW | MAF | CR | Alleles | CHR | Position | Gene | GeneID | class |
|---|---|---|---|---|---|---|---|---|---|---|
| RS2847666 | 5.04E−07 | 0.117937 | 0.287375 | 0.995864 | ‘A/G’ | 11 | 59616152 | MS4A2 | 2206 | intron |
| RS581133 | 7.74E−07 | 1.905725 | 0.289909 | 1 | ‘C/T’ | 11 | 59638882 | | | |
| RS2841959 | 8.33E−07 | 0.814549 | 0.497874 | 0.972705 | ‘C/T’ | 1 | 161324818 | | | |
| RS540170 | 1.09E−06 | 2.278205 | 0.288686 | 0.99421 | ‘A/G’ | 11 | 59636614 | | | |
| RS6492437 | 1.35E−06 | 0.120972 | 0.39234 | 0.971878 | ‘C/T’ | 13 | 89009740 | | | |
| RS11082282 | 1.51E−06 | 0.710446 | 0.032672 | 1 | ‘G/T’ | 18 | 38418935 | | | |
| RS6507488 | 1.51E−06 | 0.710446 | 0.032672 | 1 | ‘A/G’ | 18 | 38421546 | | | |
| RS11792803 | 1.56E−06 | 0.224966 | 0.079385 | 0.995037 | ‘A/G’ | 9 | 109563610 | | | |
| RS13088837 | 2.86E−06 | 0.076821 | 0.290323 | 1 | ‘A/G’ | 3 | 63434165 | SYNPR | 132204 | intron |
| RS10906283 | 3.14E−06 | 2.529734 | 0.086435 | 1 | ‘A/C’ | 10 | 13101451 | | | |
| RS10518793 | 3.32E−06 | 0.122849 | 0.015302 | 1 | ‘A/G’ | 15 | 41106723 | UBR1 | 197131 | intron |
| RS1981429 | 4.23E−06 | 0.006098 | 0.488825 | 0.999173 | ‘A/C’ | 20 | 43409107 | SDC4 | 6385 | intron |
| RS1411290 | 4.3E−06 | 0.030041 | 0.11249 | 1 | ‘A/G’ | 10 | 109589429 | | | |
| RS2841981 | 4.74E−06 | 0.019897 | 0.487572 | 0.998346 | ‘C/T’ | 1 | 161352134 | | | |
| RS16884072 | 4.94E−06 | 0.127074 | 0.208023 | 1 | ‘A/G’ | 6 | 20763482 | CDKAL1 | 54901 | intron |
| RS736425 | 4.94E−06 | 0.127074 | 0.208023 | 1 | ‘C/T’ | 6 | 20772291 | CDKAL1 | 54901 | intron |
| RS8022938 | 6.25E−06 | 7.494477 | 0.020281 | 0.999173 | ‘A/G’ | 14 | 66925080 | PLEK2 | 26499 | intron |
| RS10484632 | 6.33E−06 | 0.007833 | 0.216239 | 0.998346 | ‘A/C’ | 6 | 20755639 | CDKAL1 | 54901 | intron |
| RS4852323 | 7.71E−06 | 5.710699 | 0.306452 | 1 | ‘A/G’ | 2 | 74037181 | DGUOK | 1716 | intron |
| RS7157453 | 8.01E−06 | 0.209203 | 0.433002 | 1 | ‘C/T’ | 14 | 54228954 | SAMD4A | 23034 | intron |
| RS13194407 | 8.1E−06 | 0.002442 | 0.196443 | 1 | ‘G/T’ | 6 | 20738932 | CDKAL1 | 54901 | intron |
| RS1523558 | 8.1E−06 | 0.137015 | 0.214879 | 0.995037 | ‘A/G’ | 4 | 162222027 | | | |
| RS4686482 | 8.57E−06 | 0.391051 | 0.026882 | 1 | ‘A/G’ | 3 | 189591696 | LPP | 4026 | intron |
| RS17491334 | 9.11E−06 | 0.393652 | 0.102151 | 1 | ‘A/G’ | 12 | 5974105 | VWF | 7450 | intron |

| SNP | B1 | SE1 | P1 | n1 | B2 | SE2 | P2 | n2 | z | P-value |
|---|---|---|---|---|---|---|---|---|---|---|
| RS2847666 | −0.0027 | 0.000737 | 0.000272 | 516 | 0.002039 | 0.000589 | 0.00057856 | 537 | −5.02527 | 5.04E−07 |
| RS581133 | −0.00277 | 0.000754 | 0.000269 | 511 | 0.001933 | 0.000579 | 0.00090661 | 545 | −4.94201 | 7.74E−07 |
| RS2841959 | −0.00187 | 0.000566 | 0.001017 | 760 | 0.00308 | 0.000829 | 0.0002486 | 264 | −4.92775 | 8.33E−07 |
| RS540170 | −0.00273 | 0.000762 | 0.000367 | 505 | 0.001933 | 0.000579 | 0.00090661 | 545 | −4.87466 | 1.09E−06 |
| RS6492437 | −0.00203 | 0.000632 | 0.001379 | 653 | 0.002498 | 0.000692 | 0.000347 | 370 | −4.833 | 1.35E−06 |
| RS11082282 | 0.007037 | 0.001486 | 1.28E−05 | 63 | −0.00049 | 0.000488 | 0.31992628 | 993 | 4.810354 | 1.51E−06 |
| RS6507488 | 0.007037 | 0.001486 | 1.28E−05 | 63 | −0.00049 | 0.000488 | 0.31992628 | 993 | 4.810354 | 1.51E−06 |
| RS11792803 | −0.00555 | 0.001228 | 1.22E−05 | 153 | 0.000821 | 0.000503 | 0.10310673 | 897 | −4.80305 | 1.56E−06 |
| RS13088837 | −0.00228 | 0.000658 | 0.000584 | 517 | 0.002064 | 0.000654 | 0.00167715 | 539 | −4.68068 | 2.86E−06 |
| RS10906283 | −0.00515 | 0.001161 | 1.64E−05 | 167 | 0.000753 | 0.000507 | 0.13797872 | 889 | −4.66141 | 3.14E−06 |
| RS10518793 | −0.00958 | 0.002065 | 6.41E−05 | 30 | 0.000271 | 0.000476 | 0.56942529 | 1026 | −4.64994 | 3.32E−06 |
| RS1981429 | 0.001023 | 0.000516 | 0.048038 | 788 | −0.00443 | 0.001067 | 4.44E−05 | 268 | 4.599765 | 4.23E−06 |
| RS1411290 | −0.00433 | 0.001023 | 3.36E−05 | 226 | 0.000949 | 0.000522 | 0.06925042 | 830 | −4.59678 | 4.3E−06 |
| RS2841981 | −0.00161 | 0.000566 | 0.004445 | 777 | 0.002939 | 0.000819 | 0.00039011 | 277 | −4.57601 | 4.74E−06 |
| RS16884072 | −0.00316 | 0.000844 | 0.000209 | 393 | 0.001453 | 0.000554 | 0.00894903 | 663 | −4.56753 | 4.94E−06 |
| RS736425 | −0.00316 | 0.000844 | 0.000209 | 393 | 0.001453 | 0.000554 | 0.00894903 | 663 | −4.56753 | 4.94E−06 |
| RS8022938 | −0.01384 | 0.003092 | 5.92E−05 | 41 | 0.000288 | 0.000471 | 0.54165452 | 1014 | −4.51792 | 6.25E−06 |
| RS10484632 | −0.0031 | 0.000835 | 0.000233 | 403 | 0.001435 | 0.000559 | 0.01043089 | 651 | −4.51511 | 6.33E−06 |
| RS4852323 | 0.001642 | 0.000596 | 0.006069 | 539 | −0.00263 | 0.000746 | 0.00046152 | 517 | 4.473387 | 7.71E−06 |
| RS7157453 | 0.001179 | 0.000547 | 0.03145 | 724 | −0.00348 | 0.000887 | 0.00010901 | 332 | 4.465191 | 8.01E−06 |
| RS13194407 | −0.00329 | 0.000882 | 0.000219 | 375 | 0.001334 | 0.000545 | 0.01460502 | 681 | −4.46265 | 8.1E−06 |
| RS1523558 | −0.00239 | 0.000718 | 0.000939 | 395 | 0.001821 | 0.000613 | 0.00310207 | 656 | −4.46261 | 8.1E−06 |
| RS4686482 | −0.01128 | 0.002569 | 5.28E−05 | 54 | 0.000343 | 0.000473 | 0.46880618 | 1002 | −4.45051 | 8.57E−06 |
| RS17491334 | −0.00416 | 0.001041 | 9.14E−05 | 195 | 0.001002 | 0.000519 | 0.05382725 | 861 | −4.4375 | 9.11E−06 |

The SNPs and associated markers can be used for nutrigenetic diagnostics for the selection of individuals for low-carbohydrate food products.

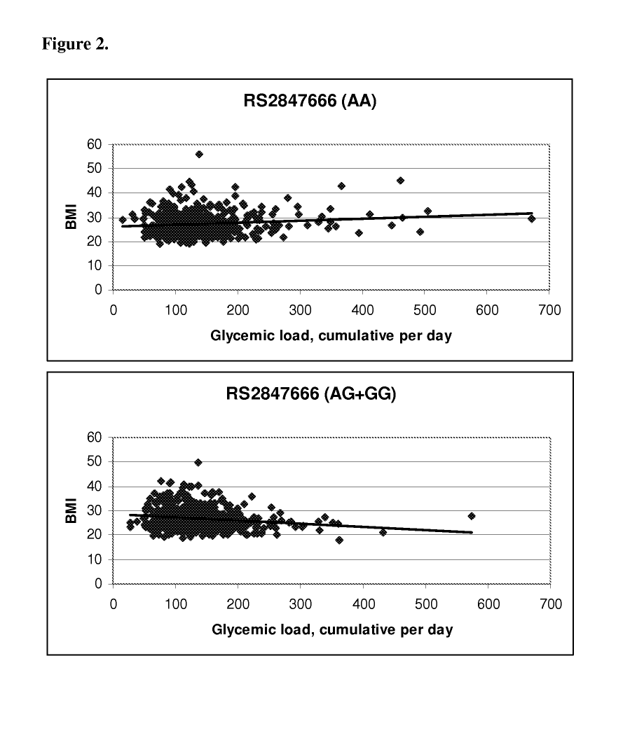

FIG. 2 shows linear regression between glycemic load and BMI for RS2847666.

The SNP rs2847666 which is located in the MS4A2 gene, modifies the effect of dietary glycemic index on BMI. Almost half of the people are major allele (A) homozygotes (upper panel), and in them a high glycemic load appears to increase BMI, while in the minor allele (G) carriers, the higher the glycemic load, the lower the BMI (lower panel).

BMI Per Glycemic Index×SNP -Interaction

TABLE 10
Results from the glycemic index × SNP -interaction for BMI.

| SNP | P-value | HW | MAF | CR | Alleles | CHR | Position | Gene | GeneID | class |
|---|---|---|---|---|---|---|---|---|---|---|
| RS10762971 | 4.92E−08 | 0.082899 | 0.012407 | 1 | ‘A/G’ | 10 | 54751285 | | | |
| RS17301783 | 3.04E−06 | 0.964084 | 0.021919 | 1 | ‘C/T’ | 1 | 60847990 | | | |
| RS9452215 | 4.15E−06 | 3.5637 | 0.096774 | 1 | ‘A/G’ | 6 | 93809954 | | | |
| RS9452223 | 4.15E−06 | 3.5637 | 0.096774 | 1 | ‘A/G’ | 6 | 93813514 | | | |
| RS830994 | 6.14E−06 | 0.267951 | 0.314309 | 1 | ‘A/G’ | 2 | 1.7E+08 | LRP2 | 4036 | coding-synonymous |
| RS6571713 | 6.31E−06 | 1.572395 | 0.069065 | 1 | ‘C/T’ | 14 | 34899460 | | | |

| SNP | B1 | SE1 | P1 | n1 | B2 | SE2 | P2 | n2 | z | P-value |
|---|---|---|---|---|---|---|---|---|---|---|
| RS10762971 | −0.12801 | 0.020656 | 2.10E−06 | 24 | −0.01218 | 0.004927 | 0.013601 | 1032 | −5.45442 | 4.92E−08 |
| RS17301783 | −0.10467 | 0.019676 | 3.15E−06 | 45 | −0.00994 | 0.004969 | 0.045822 | 1011 | −4.6682 | 3.04E−06 |
| RS9452215 | 0.028983 | 0.010241 | 0.005164 | 187 | −0.0244 | 0.005436 | 8.13E−06 | 869 | 4.604056 | 4.15E−06 |
| RS9452223 | 0.028983 | 0.010241 | 0.005164 | 187 | −0.0244 | 0.005436 | 8.13E−06 | 869 | 4.604056 | 4.15E−06 |
| RS830994 | 0.004844 | 0.00651 | 0.457179 | 575 | −0.03881 | 0.00713 | 8.32E−08 | 481 | 4.521803 | 6.14E−06 |
| RS6571713 | 0.03598 | 0.011786 | 0.002732 | 135 | −0.02231 | 0.005261 | 2.45E−05 | 921 | 4.516036 | 6.31E−06 |

The SNPs and associated markers can be used for nutrigenetic diagnostics for the selection of individuals for low-carbohydrate food products.

WHR Per Energy Intake×SNP Interaction (“Energy Intolerance”)

SUMMARY: The closest genes on chromosome 11 for the significant SNP are ANO5 (anoctamin 5, GeneID: 203859, mRNA NM 213599.2, genomic reference NC_000011.9) and NELL1 (NEL-like 1 (chicken), GeneID: 4745, mRNA NM_{—}006157.3, genomic reference NC_{—}000011.9). The significant intronic SNP is located in DNAH11 (dynein, axonemal, heavy chain 11), however the MAF of this SNP is very low thus the results are unreliable.

TABLE 11
Continuous variable: WHR adjusted for: gender, age, smoker, alcohol use, average
weekly exercise.

| SNP | P | HW | MAF | CR | CHR | Position | Gene | GeneID | class |
|---|---|---|---|---|---|---|---|---|---|
| RS10833641 | 3.59E−09 | 0.028118 | 0.492475 | 0.989247 | 11 | 21796469 | | | |
| RS4617585 | 2.31E−07 | 0.861317 | 0.443475 | 0.995037 | 11 | 21839434 | | | |
| RS7807695 | 4.84E−07 | 0.108639 | 0.015715 | 1 | 7 | 21836050 | DNAH11 | 8701 | intron |
| RS1524783 | 6.68E−07 | 0.768707 | 0.461538 | 1 | 3 | 83883488 | | | |
| RS17269759 | 9.27E−07 | 0.108639 | 0.01861 | 1 | 2 | 114269008 | | | |

| SNP | B1 | SE1 | P1 | n1 | B2 | SE2 | P2 | n2 | z | P |
|---|---|---|---|---|---|---|---|---|---|---|
| RS10833641 | −3.19E−05 | 1.06E−05 | 0.002739 | 785 | 0.000103 | 2.03E−05 | 6.83E−07 | 265 | −5.90209 | 3.59E−09 |
| RS4617585 | −2.91E−05 | 1.08E−05 | 0.007256 | 743 | 8.58E−05 | 1.94E−05 | 1.36E−05 | 313 | −5.17224 | 2.31E−07 |
| RS7807695 | 0.000178 | 3.69E−05 | 4.07E−05 | 29 | −1.38E−05 | 9.68E−06 | 0.155456 | 1032 | 5.032556 | 4.84E−07 |
| RS1524783 | −2.88E−05 | 1.07E−05 | 0.007002 | 750 | 8.29E−05 | 1.98E−05 | 3.66E−05 | 311 | −4.97034 | 6.68E−07 |
| RS17269759 | 0.000286 | 5.94E−05 | 2.45E−05 | 37 | −8.82E−06 | 9.57E−06 | 0.35731 | 1024 | 4.906586 | 9.27E−07 |

Possibly for nutrigenetic diagnostics for the selection of individuals for low-energy food products.

WHR Per Fat Intake×SNP Interaction (“Fat Intolerance”)

SUMMARY: The closest genes on chromosome 11 for the significant SNP are ANO5 (anoctamin 5) and NELL1 (NEL-like 1 (chicken)). The significant intronic SNP is located in RNF216 (ring finger protein 216; GeneID: 54476; mRNA NM_{—}207111.2, genomic reference NC_{—}000007.13). The alias for RNF216 is TRIAD3.

TABLE 12
Continuous variable: WHR adjusted for: gender, age, smoker, alcohol use, average
weekly exercise.

| SNP | P | HW | MAF | CR | CHR | Position | Gene | GeneID | class |
|---|---|---|---|---|---|---|---|---|---|
| RS10833641 | 1.2396E−08 | 0.028118 | 0.492475 | 0.989247 | 11 | 21796469 | | | |
| RS3779095 | 2.1335E−08 | 1.371882 | 0.11249 | 1 | 7 | 5701972 | TRIAD3 | 54476 | intron |
| RS4617585 | 5.1765E−08 | 0.861317 | 0.443475 | 0.995037 | 11 | 21839434 | | | |
| RS13241373 | 9.76E−08 | 0.350294 | 0.143507 | 1 | 7 | 5789747 | | | |
| RS7579789 | 6.6056E−07 | 1.11849 | 0.249379 | 0.998346 | 2 | 176319119 | | | |

| SNP | B1 | SE1 | P1 | n1 | B2 | SE2 | P2 | n2 | z | P |
|---|---|---|---|---|---|---|---|---|---|---|
| RS10833641 | −0.00242017 | 0.001216 | 0.04694 | 785 | 0.011997 | 0.00222049 | 1.46E−07 | 266 | −5.69468 | 1.24E−08 |
| RS3779095 | 0.01496209 | 0.002708 | 9.18E−08 | 222 | −0.00154 | 0.00115952 | 0.18511314 | 840 | 5.601273 | 2.13E−08 |
| RS4617585 | −0.00225763 | 0.001234 | 0.067642 | 743 | 0.011249 | 0.00215168 | 3.14E−07 | 314 | −5.44556 | 5.18E−08 |
| RS13241373 | 0.01315767 | 0.002514 | 3.23E−07 | 283 | −0.00165 | 0.00118205 | 0.16283765 | 779 | 5.331525 | 9.76E−08 |
| RS7579789 | −0.00498272 | 0.001582 | 0.001743 | 478 | 0.005676 | 0.00144572 | 9.66E−05 | 582 | −4.97291 | 6.61E−07 |

Possibly for nutrigenetic diagnostics for the selection of individuals for low-fat food products.

WHR Per Carbohydrate Intake×SNP Interaction (“Carbohydrate Intolerance”)

SUMMARY: The significant intronic SNP is located in DNAH11 (dynein, axonemal, heavy chain 11), however the MAF of this SNP is very low thus the results are unreliable. The closest genes on chromosome 11 are ANO5 (anoctamin 5) and NELL1 (NEL-like 1 (chicken)).

TABLE 13
Results from the carbohydrate intake × SNP—interaction for WHR. Continuous variable:
WHR adjusted for: gender, age, smoker, alcohol use, average weekly exercise.

| SNP | P-value | HW | MAF | CR | CHR | Position | Gene | GeneID | class |
|---|---|---|---|---|---|---|---|---|---|
| RS7807695 | 4.0127E−07 | 0.108639 | 0.015715 | 1 | 7 | 21836050 | DNAH11 | 8701 | intron |
| RS10833641 | 5.702E−07 | 0.028118 | 0.492475 | 0.989247 | 11 | 21796469 | | | |
| RS12189436 | 1.4627E−06 | 0.006052 | 0.071547 | 1 | 5 | 29518112 | | | |
| RS7937772 | 1.4977E−06 | 0.122849 | 0.016956 | 1 | 11 | 130626471 | | | |
| RS7937841 | 1.4977E−06 | 0.122849 | 0.016956 | 1 | 11 | 130626725 | | | |
| RS4298115 | 2.846E−06 | 0.386317 | 0.462366 | 1 | 4 | 47255143 | ATP10D | 57205 | intron |
| RS1524783 | 3.8855E−06 | 0.768707 | 0.461538 | 1 | 3 | 83883488 | | | |
| RS17024019 | 4.8063E−06 | 0.096262 | 0.048801 | 1 | 3 | 87340113 | | | |
| RS12026494 | 7.4556E−06 | 1.694183 | 0.045944 | 0.999173 | 1 | 207161387 | | | |
| RS6500980 | 7.6218E−06 | 0.000067 | 0.035567 | 1 | 16 | 7529121 | A2BP1 | 54715 | intron |
| RS1551310 | 8.2732E−06 | 6.665774 | 0.013659 | 0.999173 | 11 | 68613614 | | | |
| RS17023900 | 9.3168E−06 | 0.902669 | 0.039289 | 1 | 3 | 87217490 | | | |

| SNP | B1 | SE1 | P1 | n1 | B2 | SE2 | P2 | n2 | z | P-value |
|---|---|---|---|---|---|---|---|---|---|---|
| RS7807695 | 0.00557411 | 0.001211 | 7.62E−05 | 29 | −0.00074 | 0.00029517 | 0.01199334 | 1033 | 5.068675 | 4.01E−07 |
| RS10833641 | −0.00117607 | 0.000324 | 0.000304 | 785 | 0.002416 | 0.00064088 | 0.00020149 | 266 | −5.00135 | 5.7E−07 |
| RS12189436 | 0.00329891 | 0.000825 | 9.95E−05 | 151 | −0.00094 | 0.00030689 | 0.00222826 | 911 | 4.81653 | 1.46E−06 |
| RS7937772 | 0.00626318 | 0.001415 | 8.54E−05 | 36 | −0.00069 | 0.00029374 | 0.01887635 | 1026 | 4.811801 | 1.5E−06 |
| RS7937841 | 0.00626318 | 0.001415 | 8.54E−05 | 36 | −0.00069 | 0.00029374 | 0.01887635 | 1026 | 4.811801 | 1.5E−06 |
| RS4298115 | 0.00023879 | 0.000325 | 0.462561 | 754 | −0.003 | 0.00061195 | 1.48E−06 | 308 | 4.681863 | 2.85E−06 |
| RS1524783 | −0.00114262 | 0.000324 | 0.000439 | 751 | 0.002104 | 0.00062416 | 0.00084428 | 311 | −4.61764 | 3.89E−06 |
| RS17024019 | 0.00360124 | 0.000914 | 0.000156 | 95 | −0.0008 | 0.00030304 | 0.00809901 | 967 | 4.573282 | 4.81E−06 |
| RS12026494 | 0.00334836 | 0.000879 | 0.000251 | 92 | −0.00082 | 0.00030419 | 0.00727379 | 969 | 4.48044 | 7.46E−06 |
| RS6500980 | −0.00528339 | 0.001089 | 6.77E−06 | 73 | −0.00023 | 0.00029875 | 0.44524503 | 989 | −4.47573 | 7.62E−06 |
| RS1551310 | −0.01132804 | 0.002446 | 8.91E−05 | 26 | −0.00034 | 0.00029132 | 0.23664654 | 1035 | −4.45818 | 8.27E−06 |
| RS17023900 | 0.00417785 | 0.001069 | 0.000195 | 79 | −0.00074 | 0.00029928 | 0.01318617 | 983 | 4.432634 | 9.32E−06 |

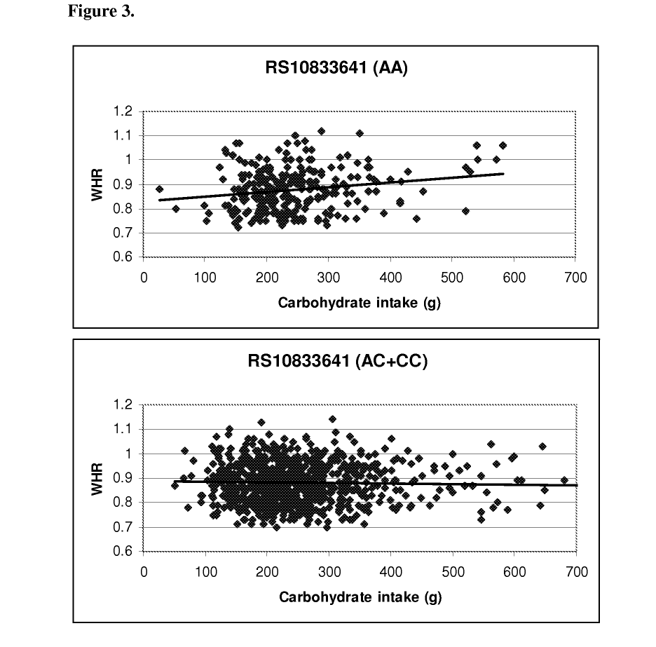

FIG. 3 shows linear regression between carbohydrate intake and WHR in RS 10833641 genotypes.

In the minor allele (A) homozygotes of rs10833641 (upper panel), the higher the carbohydrate intake, the greater the waist-to-hip circumference ratio, while in other persons, there was almost no relationship (lower panel). Can be used for nutrigenetic diagnostics for the selection of individuals for low-carbohydrate food products.

WHR Per Glycemic Load, Cumulative Per Day×SNP Interaction (“Carbohydrate Intolerance”)

SUMMARY: The significant intronic SNP is located in DNAH11 (dynein, axonemal, heavy chain 11; GeneID: 8701; mRNA NM_{—}003777.3, genomic reference NC_{—}000007.13), however the MAF of this SNP is very low thus the results are unreliable. The closest genes on chromosome 11 are ANO5 (anoctamin 5) and NELL1 (NEL-like 1 (chicken)). The closest gene on chromosome 3 is VGLL3 (vestigial like 3 (Drosophila); GeneID: 389136; mRNA NM_{—}016206.2; genomic reference NC_{—}000003.11).

TABLE 14
Results from the glycemic load × SNP - interaction for WHR. Continuous variable: WHR
adjusted for: gender, age, smoker, alcohol use, average weekly exercise

| SNP | P-value | HW | MAF | CR | Alleles | CHR | Position | Gene | GeneID | class |
|---|---|---|---|---|---|---|---|---|---|---|
| RS7807695 | 2.12E−08 | 0.108639 | 0.015715 | 1 | ‘C/T’ | 7 | 21836050 | DNAH11 | 8701 | intron |
| RS17023900 | 8.85E−08 | 0.902669 | 0.039289 | 1 | ‘A/G’ | 3 | 87217490 | | | |
| RS17024019 | 1.39E−07 | 0.096262 | 0.048801 | 1 | ‘A/G’ | 3 | 87340113 | | | |
| RS10833641 | 2.88E−07 | 0.028118 | 0.492475 | 0.989247 | ‘A/C’ | 11 | 21796469 | | | |
| RS2837958 | 8.08E−07 | 0.259261 | 0.328371 | 1 | ‘A/G’ | 21 | 41413983 | | | |
| RS7937772 | 8.62E−07 | 0.122849 | 0.016956 | 1 | ‘A/G’ | 11 | 130626471 | | | |
| RS7937841 | 8.62E−07 | 0.122849 | 0.016956 | 1 | ‘C/T’ | 11 | 130626725 | | | |
| RS4298115 | 1.14E−06 | 0.386317 | 0.462366 | 1 | ‘C/T’ | 4 | 47255143 | ATP10D | 57205 | intron |
| RS2173199 | 1.34E−06 | 3.190972 | 0.479322 | 1 | ‘A/G’ | 4 | 100390402 | | | |
| RS6532814 | 1.34E−06 | 3.190972 | 0.479322 | 1 | ‘C/T’ | 4 | 100392991 | | | |
| RS12189436 | 1.54E−06 | 0.006052 | 0.071547 | 1 | ‘A/G’ | 5 | 29518112 | | | |
| RS2837957 | 1.87E−06 | 0.000843 | 0.352766 | 0.971878 | ‘C/T’ | 21 | 41412990 | | | |
| RS12510722 | 1.96E−06 | 2.916783 | 0.478785 | 0.99421 | ‘A/G’ | 4 | 100366124 | | | |
| RS12396657 | 2.09E−06 | 102.6038 | 0.015315 | 0.999173 | ‘C/T’ | X | 52020437 | | | |
| RS241541 | 2.6E−06 | 0.315918 | 0.056291 | 0.999173 | ‘A/C’ | 14 | 55578421 | | | |
| RS10503080 | 2.66E−06 | 0.153971 | 0.014061 | 1 | ‘C/T’ | 18 | 59285262 | | | |
| RS15362 | 2.79E−06 | 0.100615 | 0.145575 | 1 | ‘C/T’ | 17 | 1370316 | PITPNA | 5306 | mrna-utr |
| RS10497546 | 3.97E−06 | 0.207476 | 0.017783 | 1 | ‘C/T’ | 2 | 180228875 | ZNF533 | 151126 | intron |
| RS2276572 | 3.97E−06 | 0.207476 | 0.017783 | 1 | ‘A/G’ | 2 | 180250807 | ZNF533 | 151126 | intron |
| RS7570893 | 3.97E−06 | 0.207476 | 0.017783 | 1 | ‘C/T’ | 2 | 180273415 | ZNF533 | 151126 | intron |
| RS3107864 | 3.99E−06 | 0.872878 | 0.23234 | 0.971878 | ‘A/G’ | 2 | 73991352 | ACTG2 | 72 | intron |
| RS12026494 | 4.25E−06 | 1.694183 | 0.045944 | 0.999173 | ‘A/C’ | 1 | 207161387 | | | |
| RS5943662 | 4.48E−06 | 102.6038 | 0.015715 | 1 | ‘C/T’ | X | 52016710 | | | |
| RS1524783 | 4.71E−06 | 0.768707 | 0.461538 | 1 | ‘G/T’ | 3 | 83883488 | | | |
| RS17362588 | 4.78E−06 | 0.426472 | 0.066694 | 0.998346 | ‘A/G’ | 2 | 179429291 | FLJ39502 | 285025 | reference |
| RS10251790 | 5.4E−06 | 2.878055 | 0.212801 | 0.995037 | ‘C/T’ | 7 | 12064226 | | | |
| RS4891429 | 5.73E−06 | 0.026799 | 0.010753 | 1 | ‘A/C’ | 18 | 66989691 | | | |
| RS2776340 | 5.8E−06 | 0.095958 | 0.336348 | 0.960298 | ‘A/G’ | 21 | 41364420 | | | |
| RS1653257 | 6.99E−06 | 0.000725 | 0.114144 | 1 | ‘C/T’ | 2 | 73982413 | ACTG2 | 72 | intron |
| RS2600933 | 7.25E−06 | 0.291742 | 0.021092 | 1 | ‘A/G’ | 12 | 41210570 | PRICKLE1 | 144165 | intron |
| RS4379440 | 8.2E−06 | 0.188725 | 0.01861 | 1 | ‘G/T’ | 8 | 62628368 | ASPH | 444 | intron |
| RS11074063 | 8.23E−06 | 0.989614 | 0.112169 | 0.999173 | ‘A/G’ | 15 | 90688982 | | | |
| RS6537639 | 8.3E−06 | 2.83465 | 0.18543 | 0.999173 | ‘C/T’ | 22 | 48975813 | TRABD | 80305 | intron |
| RS340639 | 8.39E−06 | 0.019784 | 0.28146 | 0.985939 | ‘A/G’ | 4 | 88144003 | | | |
| RS2271253 | 8.95E−06 | 1.285949 | 0.01861 | 1 | ‘C/T’ | 18 | 59305331 | SERPINB5 | 5268 | intron |
| RS9967149 | 8.95E−06 | 1.285949 | 0.01861 | 1 | ‘A/G’ | 18 | 59302179 | SERPINB5 | 5268 | intron |
| RS5991847 | 9.34E−06 | 71.47708 | 0.013256 | 0.998346 | ‘C/T’ | X | 53041679 | | | |
| RS1393851 | 9.45E−06 | 0.546943 | 0.096761 | 0.995864 | ‘C/T’ | 4 | 167082335 | TLL1 | 7092 | intron |

| SNP | B1 | SE1 | P1 | n1 | B2 | SE2 | P2 | n2 | z | P-value |
|---|---|---|---|---|---|---|---|---|---|---|
| RS7807695 | 0.008832 | 0.001769 | 2.58E−05 | 29 | −0.00146 | 0.000498 | 0.00338357 | 1033 | 5.602533 | 2.12E−08 |
| RS17023900 | 0.007471 | 0.001599 | 1.21E−05 | 79 | −0.0015 | 0.000504 | 0.00302063 | 983 | 5.349276 | 8.85E−08 |
| RS17024019 | 0.006617 | 0.001467 | 1.85E−05 | 95 | −0.00156 | 0.000509 | 0.00217908 | 967 | 5.267484 | 1.39E−07 |
| RS10833641 | −0.00227 | 0.000554 | 4.62E−05 | 785 | 0.00356 | 0.000992 | 0.00039388 | 266 | −5.13179 | 2.88E−07 |
| RS2837958 | −0.00311 | 0.000665 | 3.57E−06 | 595 | 0.001625 | 0.000692 | 0.01931445 | 467 | −4.93369 | 8.08E−07 |
| RS7937772 | 0.008871 | 0.002021 | 9.51E−05 | 36 | −0.00137 | 0.000496 | 0.00590497 | 1026 | 4.921002 | 8.62E−07 |
| RS7937841 | 0.008871 | 0.002021 | 9.51E−05 | 36 | −0.00137 | 0.000496 | 0.00590497 | 1026 | 4.921002 | 8.62E−07 |
| RS4298115 | 0.000402 | 0.000552 | 0.466414 | 754 | −0.00506 | 0.000976 | 4.05E−07 | 308 | 4.865955 | 1.14E−06 |
| RS2173199 | −0.00211 | 0.000549 | 0.000137 | 794 | 0.003427 | 0.001004 | 0.00073955 | 268 | −4.83466 | 1.34E−06 |
| RS6532814 | −0.00211 | 0.000549 | 0.000137 | 794 | 0.003427 | 0.001004 | 0.00073955 | 268 | −4.83466 | 1.34E−06 |
| RS12189436 | 0.005144 | 0.001336 | 0.000174 | 151 | −0.00174 | 0.000517 | 0.00079327 | 911 | 4.806119 | 1.54E−06 |
| RS2837957 | −0.00288 | 0.000657 | 1.38E−05 | 605 | 0.001828 | 0.000737 | 0.01352219 | 433 | −4.76689 | 1.87E−06 |
| RS12510722 | −0.00205 | 0.000549 | 0.000206 | 786 | 0.003396 | 0.001004 | 0.00082286 | 269 | −4.75724 | 1.96E−06 |
| RS12396657 | 0.009414 | 0.002199 | 0.000258 | 24 | −0.00128 | 0.000493 | 0.00977542 | 1037 | 4.744683 | 2.09E−06 |
| RS241541 | 0.005976 | 0.001513 | 0.000139 | 109 | −0.00153 | 0.000509 | 0.00276606 | 952 | 4.700589 | 2.6E−06 |
| RS10503080 | 0.008827 | 0.002087 | 0.000202 | 30 | −0.00124 | 0.000494 | 0.01205977 | 1032 | 4.695635 | 2.66E−06 |
| RS15362 | 0.002969 | 0.000946 | 0.001868 | 290 | −0.00218 | 0.00056 | 0.00010662 | 772 | 4.686226 | 2.79E−06 |
| RS10497546 | −0.01075 | 0.002174 | 1.49E−05 | 39 | −0.00046 | 0.000494 | 0.34827351 | 1023 | −4.61299 | 3.97E−06 |
| RS2276572 | −0.01075 | 0.002174 | 1.49E−05 | 39 | −0.00046 | 0.000494 | 0.34827351 | 1023 | −4.61299 | 3.97E−06 |
| RS7570893 | −0.01075 | 0.002174 | 1.49E−05 | 39 | −0.00046 | 0.000494 | 0.34827351 | 1023 | −4.61299 | 3.97E−06 |
| RS3107864 | 0.002113 | 0.000785 | 0.00739 | 417 | −0.00249 | 0.000616 | 6.02E−05 | 616 | 4.61219 | 3.99E−06 |
| RS12026494 | 0.004612 | 0.001256 | 0.000404 | 92 | −0.00164 | 0.00052 | 0.00164915 | 969 | 4.599114 | 4.25E−06 |
| RS5943662 | 0.009187 | 0.002227 | 0.000358 | 25 | −0.00128 | 0.000493 | 0.00977542 | 1037 | 4.588132 | 4.48E−06 |
| RS1524783 | −0.00211 | 0.000549 | 0.000138 | 751 | 0.003119 | 0.001 | 0.00198905 | 311 | −4.57767 | 4.71E−06 |
| RS17362588 | 0.004744 | 0.001315 | 0.000429 | 142 | −0.00173 | 0.000521 | 0.00094125 | 918 | 4.574537 | 4.78E−06 |
| RS10251790 | 0.002397 | 0.00092 | 0.009488 | 396 | −0.00251 | 0.000563 | 9.84E−06 | 661 | 4.548724 | 5.4E−06 |
| RS4891429 | 0.011148 | 0.002669 | 0.000465 | 20 | −0.00116 | 0.000489 | 0.01771649 | 1042 | 4.536444 | 5.73E−06 |
| RS2776340 | −0.0031 | 0.000697 | 1.04E−05 | 568 | 0.001514 | 0.000742 | 0.04177989 | 452 | −4.53362 | 5.8E−06 |
| RS1653257 | 0.003608 | 0.0011 | 0.001202 | 233 | −0.00189 | 0.000536 | 0.00043575 | 829 | 4.494109 | 6.99E−06 |
| RS2600933 | 0.008556 | 0.002152 | 0.000324 | 36 | −0.00135 | 0.000494 | 0.00632681 | 1026 | 4.48647 | 7.25E−06 |
| RS4379440 | 0.008133 | 0.002053 | 0.000445 | 29 | −0.00129 | 0.000495 | 0.00943464 | 1033 | 4.460212 | 8.2E−06 |
| RS11074063 | 0.003436 | 0.001061 | 0.001381 | 226 | −0.00188 | 0.000543 | 0.00056877 | 835 | 4.459319 | 8.23E−06 |
| RS6537639 | −0.00416 | 0.000876 | 2.94E−06 | 353 | 0.000515 | 0.000578 | 0.37281123 | 709 | −4.4574 | 8.3E−06 |
| RS340639 | 0.001447 | 0.000706 | 0.040963 | 513 | −0.00288 | 0.000666 | 1.86E−05 | 534 | 4.455203 | 8.39E−06 |
| RS2271253 | 0.006631 | 0.001709 | 0.000381 | 40 | −0.00128 | 0.0005 | 0.01080464 | 1022 | 4.441201 | 8.95E−06 |
| RS9967149 | 0.006631 | 0.001709 | 0.000381 | 40 | −0.00128 | 0.0005 | 0.01080464 | 1022 | 4.441201 | 8.95E−06 |
| RS5991847 | 0.009205 | 0.002313 | 0.000591 | 23 | −0.00128 | 0.000493 | 0.00978971 | 1038 | 4.43219 | 9.34E−06 |
| RS1393851 | 0.002844 | 0.000939 | 0.002823 | 185 | −0.002 | 0.000558 | 0.00036475 | 873 | 4.429487 | 9.45E−06 |

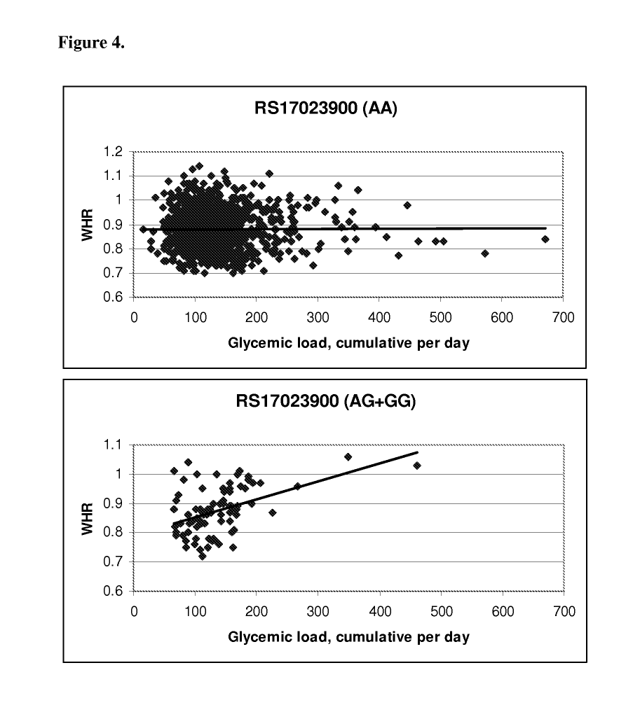

FIG. 4 shows linear regression between glycemic load and WHR in RS 17023900 genotypes.

In the major allele (A) homozygotes (upper panel), there was no relationship between the glycemic load and WHR, while in the minor allele (G) carriers, there was a strong direct association between the glycemic index and WHR (lower panel), though the upward linear slope was to a large degree due to four extreme individuals.

WHR Per Glycemic Index×SNP -Interaction

TABLE 15
Results from the glycemic index × SNP - interaction for WHR.

| SNP | P-value | HW | MAF | CR | Alleles | CHR | Position | Gene | GeneID | class |
|---|---|---|---|---|---|---|---|---|---|---|
| RS3731572 | 1.57E−07 | 0.988079 | 0.035153 | 1 | ‘A/C’ | 1 | 14448579 | | | |
| RS9614978 | 2.71E−07 | 12.86182 | 0.051839 | 0.989247 | ‘C/T’ | 4 | 26401738 | | | |
| RS11750694 | 3.2E−07 | 0.526267 | 0.1555 | 1 | ‘A/C’ | 5 | 29059061 | | | |
| RS2588498 | 6.43E−07 | 0.546506 | 0.163772 | 1 | ‘A/C’ | 2 | 75562668 | | | |
| RS13085233 | 1.16E−06 | 0.710446 | 0.025641 | 1 | ‘A/G’ | X | 104648231 | IL1RAPL2 | 26280 | intron |
| RS12523586 | 2.46E−06 | 0.001174 | 0.129005 | 0.980976 | ‘A/G’ | 5 | 28782832 | | | |
| RS11113910 | 3.55E−06 | 0.016633 | 0.080216 | 0.995037 | ‘A/C’ | 12 | 107398793 | | | |
| RS11130760 | 4.99E−06 | 0.146223 | 0.134328 | 0.997519 | ‘A/G’ | 14 | 45057446 | | | |
| RS4910323 | 5.36E−06 | 0.782292 | 0.390728 | 0.999173 | ‘G/T’ | 11 | 11317610 | GALNTL4 | 374378 | intron |
| RS2393012 | 5.47E−06 | 0.146785 | 0.163079 | 0.999173 | ‘A/G’ | 10 | 57654600 | | | |
| RS2462466 | 5.6E−06 | 0.146785 | 0.162666 | 0.999173 | ‘C/T’ | 10 | 57668467 | | | |
| RS8179521 | 6.55E−06 | 1.941772 | 0.020678 | 1 | ‘A/G’ | 2 | 127867394 | | | |
| RS1999088 | 6.72E−06 | 1.301204 | 0.065343 | 1 | ‘C/T’ | 1 | 183911286 | | | |
| RS6581525 | 7.4E−06 | 1.470222 | 0.431291 | 0.999173 | ‘A/G’ | 1 | 183895507 | | | |
| RS12764885 | 7.44E−06 | 0.407762 | 0.160833 | 0.992556 | ‘G/T’ | 10 | 57648974 | | | |
| RS10743430 | 7.52E−06 | 0.364797 | 0.026055 | 1 | ‘C/T’ | 13 | 96725605 | MBNL2 | 10150 | intron |
| RS529674 | 7.65E−06 | 0.013152 | 0.229529 | 1 | ‘A/G’ | 1 | 53799468 | GLIS1 | 148979 | intron |
| RS1071905 | 8.57E−06 | 0.091291 | 0.457816 | 1 | ‘A/G’ | 12 | 62707815 | SRGAP1 | 57522 | intron |
| RS11796366 | 8.8E−06 | 193.4444 | 0.011185 | 0.998346 | ‘C/T’ | 12 | 22042840 | | | |
| RS17338297 | 9.1E−06 | 0.588019 | 0.144334 | 1 | ‘A/G’ | 18 | 48044577 | | | |
| RS2969018 | 9.13E−06 | 0.608208 | 0.188172 | 1 | ‘C/T’ | 7 | 2606667 | IQCE | 23288 | intron |

| SNP | B1 | SE1 | P1 | n1 | B2 | SE2 | P2 | n2 | z | P-value |
|---|---|---|---|---|---|---|---|---|---|---|
| RS3731572 | 0.080834 | 0.016808 | 7.92E−06 | 73 | −0.01159 | 0.005299 | 0.02893242 | 989 | 5.244392 | 1.57E−07 |
| RS9614978 | 0.063377 | 0.013637 | 1.02E−05 | 101 | −0.01213 | 0.005441 | 0.02596461 | 950 | 5.142892 | 2.71E−07 |
| RS11750694 | −0.04369 | 0.00892 | 1.59E−06 | 298 | 0.011643 | 0.006132 | 0.05795732 | 764 | −5.11185 | 3.2E−07 |
| RS2588498 | −0.04486 | 0.009144 | 1.49E−06 | 318 | 0.009807 | 0.006081 | 0.1072186 | 744 | −4.97805 | 6.43E−07 |
| RS13085233 | 0.098913 | 0.022085 | 4.50E−05 | 49 | −0.01141 | 0.005203 | 0.02848717 | 1013 | 4.862341 | 1.16E−06 |
| RS12523586 | −0.04759 | 0.010267 | 5.86E−06 | 240 | 0.008079 | 0.005847 | 0.16744157 | 801 | −4.71132 | 2.46E−06 |
| RS11113910 | −0.06012 | 0.012752 | 5.19E−06 | 162 | 0.004339 | 0.005537 | 0.43344574 | 895 | −4.63661 | 3.55E−06 |
| RS11130760 | 0.035935 | 0.010401 | 0.000637 | 275 | −0.01847 | 0.005815 | 0.00155352 | 784 | 4.565452 | 4.99E−06 |
| RS4910323 | −0.023 | 0.006291 | 0.000276 | 677 | 0.025537 | 0.008615 | 0.00322326 | 384 | −4.55024 | 5.36E−06 |
| RS2393012 | −0.04235 | 0.00938 | 8.96E−06 | 315 | 0.00836 | 0.006037 | 0.16653522 | 747 | −4.54595 | 5.47E−06 |
| RS2462466 | −0.04235 | 0.009395 | 9.24E−06 | 314 | 0.00836 | 0.006037 | 0.16653522 | 747 | −4.5412 | 5.6E−06 |
| RS8179521 | −0.10657 | 0.022828 | 3.72E−05 | 38 | −0.00102 | 0.005198 | 0.84401728 | 1024 | −4.50814 | 6.55E−06 |
| RS1999088 | 0.04861 | 0.012713 | 0.000204 | 129 | −0.01376 | 0.005501 | 0.01254447 | 933 | 4.502605 | 6.72E−06 |
| RS6581525 | 0.009492 | 0.006067 | 0.118137 | 729 | −0.04012 | 0.009259 | 1.95E−05 | 332 | 4.482168 | 7.4E−06 |
| RS12764885 | −0.04239 | 0.009583 | 1.35E−05 | 307 | 0.00836 | 0.006037 | 0.16653522 | 747 | −4.48097 | 7.44E−06 |
| RS10743430 | 0.07304 | 0.017972 | 0.000152 | 56 | −0.01085 | 0.00528 | 0.04008533 | 1006 | 4.478673 | 7.52E−06 |
| RS529674 | −0.03083 | 0.007551 | 5.32E−05 | 433 | 0.014803 | 0.006851 | 0.03109997 | 629 | −4.47501 | 7.65E−06 |
| RS1071905 | 0.008403 | 0.005901 | 0.154877 | 749 | −0.04271 | 0.009853 | 1.97E−05 | 313 | 4.450572 | 8.57E−06 |
| RS11796366 | 0.168609 | 0.039349 | 0.000651 | 15 | −0.00778 | 0.005133 | 0.13009609 | 1045 | 4.444892 | 8.8E−06 |
| RS17338297 | 0.033229 | 0.009931 | 0.000927 | 295 | −0.01806 | 0.00591 | 0.00232618 | 767 | 4.437696 | 9.1E−06 |
| RS2969018 | −0.03794 | 0.008864 | 2.41E−05 | 352 | 0.01003 | 0.00619 | 0.10562459 | 710 | −4.43706 | 9.13E−06 |

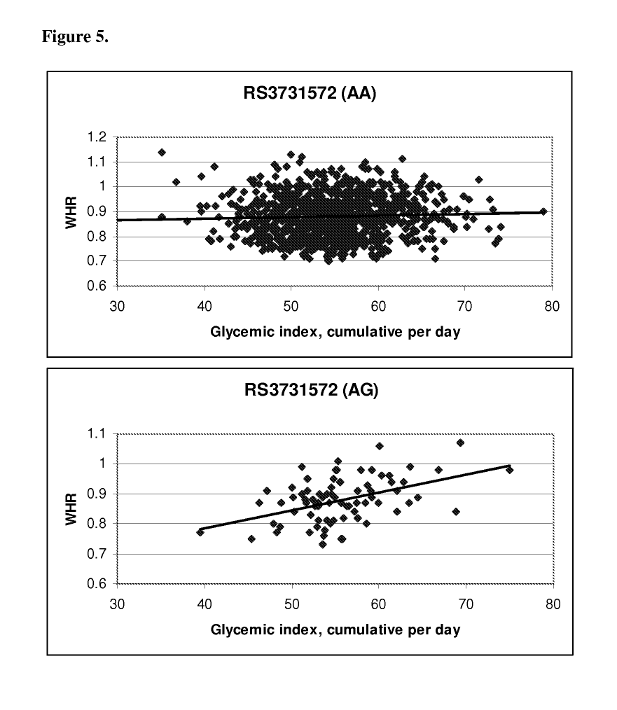

FIG. 5 shows linear regression between glycemic index and WHR in RS3731572 genotypes.

In the major allele (A) homozygotes (upper panel), there was only a weak association between the dietary glycemic index and WHR, whereas in the minor allele (G) carriers the glycemic index had a strong association with WHR (lower panel).

BMI/Carbohydrate Intake

TABLE 16
Results from the carbohydrate intolerance (BMI/carbohydrate intake).

| MARKER | N | BETA | P-value | HW | MAF | CR | CHR | POSITION | GENE | GENE_ID | CLASS |
|---|---|---|---|---|---|---|---|---|---|---|---|
| RS735984 | 1065 | 0.3124 | 1.79E−06 | 8.30 | 0.12 | 1.0000 | 11 | 43220897 | | | |
| RS1021797 | 1065 | 0.4191 | 2.74E−06 | 0.01 | 0.06 | 1.0000 | 12 | 26457699 | ITPR2 | 3709 | intron |
| RS2305299 | 1065 | 0.288 | 2.92E−06 | 0.50 | 0.14 | 1.0000 | 10 | 45456999 | ANUBL1 | 93550 | intron |
| RS9858834 | 1065 | 0.3255 | 3.45E−06 | 0.01 | 0.10 | 1.0000 | 3 | 176771673 | NAALADL2 | 254827 | intron |
| RS436488 | 1065 | 0.2638 | 3.60E−06 | 0.12 | 0.17 | 1.0000 | 19 | 61056102 | NLRP4 | 147945 | intron |
| RS12030971 | 1065 | 0.2027 | 3.72E−06 | 0.03 | 0.40 | 1.0000 | 1 | 69203851 | | | |
| RS16825963 | 1059 | 0.323 | 5.12E−06 | 0.00 | 0.10 | 0.9950 | 3 | 176764272 | NAALADL2 | 254827 | intron |
| RS7780636 | 1065 | 0.4089 | 7.81E−06 | 6.20 | 0.05 | 1.0000 | 7 | 70664399 | WBSCR17 | 64409 | intron |
| RS11633874 | 1055 | −0.1915 | 8.57E−06 | 0.04 | 0.48 | 0.9901 | 15 | 66719213 | CORO2B | 10391 | intron |
| RS1005316 | 1065 | 0.2257 | 9.40E−06 | 0.16 | 0.22 | 1.0000 | 17 | 66501964 | | | |

WHR/Carbohydrate Intake

TABLE 17
Results from the carbohydrate intolerance (WHR/carbohydrate intake)

| MARKER | N | BETA | P-value | HW | MAF | CR | CHR | POSITION | GENE | GENE_ID | CLASS |
|---|---|---|---|---|---|---|---|---|---|---|---|
| RS9858834 | 1065 | 0.3377 | 1.45E−06 | 0.01 | 0.10 | 1.0000 | 3 | 176771673 | NAALADL2 | 254827 | intron |
| RS16825963 | 1059 | 0.3325 | 2.70E−06 | 0.00 | 0.10 | 0.9950 | 3 | 176764272 | NAALADL2 | 254827 | intron |
| RS1426499 | 1065 | 0.2031 | 2.80E−06 | 1.01 | 0.41 | 1.0000 | 7 | 131885330 | PLXNA4B | 91584 | intron |
| RS2305299 | 1065 | 0.2862 | 3.36E−06 | 0.50 | 0.14 | 1.0000 | 10 | 45456999 | ANUBL1 | 93550 | intron |
| RS4442796 | 1065 | 0.2853 | 3.54E−06 | 0.25 | 0.15 | 1.0000 | 16 | 68345609 | NOB1 | 28987 | intron |
| RS4816047 | 1065 | 0.1974 | 6.18E−06 | 0.24 | 0.41 | 1.0000 | 20 | 8073220 | PLCB1 | 23236 | intron |
| RS2917682 | 1065 | 0.2794 | 6.80E−06 | 0.15 | 0.14 | 1.0000 | 16 | 68323792 | | | |

Further Genes of the Invention

DNAH11 as Energy and Carbohydrate Intolerance Gene

Data for DNAH11 Association:

Analysis of WHR by Energy_intake by SNP_interactions

Finding: SNPs in the DNAH11 gene modify the association between energy intake and glucose load and WHR.

SNP Energy intake interaction for WHR

Energy intake from Food survey

Dependent Variable: WHR residual

Regression model WHR=Energy intake

Model was separately used for samples with minor allele present and samples that are homozygous for wild (major) allele

Adjusted variables were Gender, Smoker, Age, Alcohol use, absolute ethanol grams/day,

Average weekly exercise (hours).

TABLE 18
The most significant marker.

| SNP | P | HW | MAF | CR | CHR | Position | Gene | GeneID | class |
|---|---|---|---|---|---|---|---|---|---|
| RS7807695 | 4.84E−07 | 0.108639 | 0.015715 | 1 | 7 | 21836050 | DNAH11 | 8701 | intron |

There are 160 SNPs for DNAH11 gene on Illumina 500K.

TABLE 19
All significant markers intragenic for DNAH11 in
WHR_Energy_intake_SNP_interaction analysis:

| SNP | P | HW | MAF | CR | Alleles | CHR | Position | Gene | GeneID | class |
|---|---|---|---|---|---|---|---|---|---|---|
| RS4722054 | 0.000639 | 14.09977 | 0.027709 | 1 | ‘A/G’ | 7 | 21773017 | DNAH11 | 8701 | intron |
| RS10268330 | 0.000639 | 14.09977 | 0.027709 | 1 | ‘A/G’ | 7 | 21774561 | DNAH11 | 8701 | intron |
| RS7807695 | 4.84E−07 | 0.108639 | 0.015715 | 1 | ‘C/T’ | 7 | 21836050 | DNAH11 | 8701 | intron |

R² of less significant SNPs wrt RS7807695:

| SNP | R² in 550K EF data | R² in HapMap_CEU data |
|---|---|---|
| RS4722054 | 0.011 | 0.186 |
| RS10268330 | 0.011 | 0.008 |

No high R2 SNPs in 550k assay for RS7807695 (R2_Max_EF=0.026, RS6954331)

The lowest P-values for DNAH11 in BMI/WHR analyses:

| | subjects | SNP | P |
|---|---|---|---|
| BMI Analysis | | | |
| BMI analysis | Eastern Finnish women | RS10950880 | 0.001972 |
| Min_P_BMI_{—} | | RS7807695 | |
| MEN | | RS7807695 | 0.4 |
| WHR Analysis | | | |
| WHR analyses | P_VE_MEN_WHR | RS6978629 | 0.002983 |
| WHR_RS7807695 | P_VE_WOMEN_WHR | RS7807695 | 0.01529 |

TABLE 20
Glucose load interaction.

| SNP | n1 | n2 | P | MAF | Alleles | Gene | GeneID | class |
|---|---|---|---|---|---|---|---|---|
| RS7807695 | 29 | 1033 | 2.12E−08 | 0.015715 | ‘C/T’ | DNAH11 | 8701 | intron |

The regression coefficient is positive in the smaller group and negative in the larger group. In the carriers of the minor allele a high glucose load is strongly associated with WHR, while in the others there is a weak inverse association.

RS7807695

DNAH11 is a large gene in chromosome 7 at position 21836050 bp. The marker is located in the intron 65 of DNAH11 gene.

MAF in HapMap_CEU population: 0.092 for minor allele ‘C’. MAF in EF population: 0.015715.

Our data set included 29 individuals with heterozygote minor allele genotype. No minor allele homozygotes were observed.

LD Block Structure of DNAH11 Region: Hapmap_CEU population chr7:21353669-22103668 by (750 kb)

RS7807695 is in weak linkage in HapMap CEU population with other SNPs within 750 kb window (including the neighboring genes SP4 and CDCA7L). RS7807695 has D′=1 with 208 SNPs, however, the highest R2 values are:

| marker1 | marker2 | D′ | r\hat{ }2 | LOD | Location |
|---|---|---|---|---|---|
| rs7807695 | rs2893060 | 0.662 | 0.328 | 5.01 | DNAH11 intron 74 |
| rs7807695 | rs17145742 | 0.633 | 0.322 | 4.63 | DNAH11 intron 70 |
| rs7807695 | rs4392794 | 0.682 | 0.285 | 4.97 | DNAH11 intron 76 |
| rs7807695 | rs2074329 | 0.588 | 0.264 | 4.36 | DNAH11 intron 71 |
| rs7807695 | rs1139224 | 0.769 | 0.251 | 4.63 | DNAH11 intron 79 |
| rs7807695 | rs17145715 | 0.584 | 0.241 | 4.09 | DNAH11 intron 68 |
| rs7807695 | rs10269223 | 0.545 | 0.204 | 3.31 | DNAH11 intron 75 |

The highest R² of RS7807695 in HapMap CEU population to neighboring genes is R²=0.082 to rs10238945 (CDCA7L intron).

Conclusions About the Data:

The associated SNP RS7807695 pinpoints to the gene DNAH11, in addition there are two other markers (RS4722054 and RS 10268330) in this large gene that are hits in the analysis.

DNAH11 GeneID: 8701

DNAH11 and Obesity:

DNAH11 encodes for a dynein heavy chain family protein that is a microtubule-dependent motor ATPase and participates in motility of flagella and cilia. DNAH11 is not presently known to play any role in intracellular dynein function. DNAH11 is expressed in tissues that have flagella or cilia. DNAH11 has been shown in human to associate to disorders involving perturbed or absent beating of primary motile cilia, such as in PCD and KS. The disorders are characterized by respiratory infections, reduced fertility, and situs inversus, due to dysfunction of monocilia at the embryonic node and randomization of left-right body asymmetry.

Until recently, ciliary structures were thought to be present mainly in structures with dense ciliary content, for example in epithelial lining of lungs and ear, olfactory cells, in spermatozoa, and ovaries. Recent studies have greatly increased understanding of ciliary function in several cell types and tissues. For example, in brain cilia play roles in Hedgehog-signaling, and in neural stem cell generation (Hedgehog signaling and primary cilia are required for the formation of adult neural stem cells. Nat Neurosci. 2008 Mar;11(3):277-84. PMID: 18297065).

Cilia seem to play a role in obesity, mainly based on the evidence that genes mutated in patients with BBS encode for proteins that have ciliary function. In animal models, ciliary disruption has been shown to result in obesity, potentially through central nervous system action. It has been proposed that pro-opiomelanocortin expressing cells in hypothalamus could relay the pathways for regulating satiety responses. Other locations for ciliary dysfunction in obesity are also likely.

DNAH11 appears to mainly relate to motile cilia which seem to have functions somewhat different from immotile, sensory primary cilia. Motile cilia are found in great numbers on the surface of the epithelial cells lining the airways and reproductive tracts and on epithelial cells of the ependyma and choroid plexus in the brain. DNAH11 has been especially shown to affect the motility of airway epithelial motile cilia, whereas it has been shown not to inherently affect the motility of sperm. The function of DNAH11 outside of motile cilia has not been explored.

Shah et al. have recently shown that loss of Bardet-Biedl syndrome proteins (that relate to obesity) alters the morphology and function of motile cilia in airway epithelia (Shah A S, et al. Loss of Bardet-Biedl syndrome proteins alters the morphology and function of motile cilia in airway epithelia. Proc Natl Acad Sci USA. 2008 Mar 4;105(9):3380-5. PMID: 18299575). Therefore, it is possible that also DNAH11 may play a similar role in ciliary disorders as what has been shown for BBS proteins. Moreover, although cilia are broadly classified as 9+2 type motile cilia and 9+0 type sensory immotile cilia, there are examples of 9+2 sensory cilia and 9+0 motile cilia (reviewed in Bisgrove B W, Yost H J. The roles of cilia in developmental disorders and disease. Development. 2006 Nov;133(21):4131-43. PMID: 17021045; and in Christensen ST et al., Sensory Cilia and Integration of Signal Transduction in Human Health and Disease. Traffic. 2007 Feb;8(2):97-109.). Several signaling class receptors have been located in motile cilia, including receptor tyrosine kinases, Hedgehog, Wnt and steroid signaling and ion channel/calcium signaling (reviewed in Christensen ST et al., 2007). As research with the subject is currently in heavy progress more signaling pathways in cilia are likely to be reported, and may well relate to obesity-associated mechanisms.

As a mechanism of obesity, appetite or absorption of nutrients are possibilities with relation to ciliary mechanism of obesity. Choroid plexus, the area on the ventricles of the brain where cerebrospinal fluid (CSF) is produced by modified ependymal cells, is a central site with motile cilia in the human brain. There are four choroid plexus in the brain, one in each of the ventricles. Choroid plexus is immunoreactive for leptin protein (Couce M E, et al., Localization of leptin receptor in the human brain. Neuroendocrinology. 1997 September;66(3):145-50.), and circulating leptin is transported into the brain by binding to megalin at the choroid plexus epithelium (Dietrich M O, et al. Megalin mediates the transport of leptin across the blood-CSF barrier. Neurobiol Aging. 2008 June;29(6):902-12. PMID: 17324488). Furthermore, in a mouse model interference of normal energy homeostasis by disrupting cilia on neurons throughout the central nervous system and on pro-opiomelanocortin-expressing cells in the hypothalamus (lining next to ventricular choroid plexus, resulted in obesity (Davenport J R et al., Curr Biol. 2007 Sep. 18;17(18):1586-94).

In conclusion, DNAH11 may play a role in obesity and energy and carbohydrate intolerance by modulating the function of motile cilia. This could be due to alterations for example in ciliary beating, protein transport or localization in cilia. These alterations may affect chemosensory mechanisms and/or intracellular or neuroendocrine signaling. Potential sites of action are both peripheral and central.

Markers in the DNAH11 gene also significantly modified the association between dietary glucose load and WHR (2.12×10⁻⁸). This enzyme-coding gene is also associated with obesity, T2D and CHD in several of our studies. A large proportion of individuals are susceptible to obesity because of high carbohydrate intake, they are carbohydrate intolerant. This intolerance can theoretically be cured/attenuated by functional foods against this target or its binding or functional partners.

CDKAL1 as Energy and Carbohydrate Intolerance Gene

BMI: Carbohydrate Intake×SNP Interaction

Continuous Variable: ln(BMI)

Adjusted for: Age, HT-status, average weekly exercise

TABLE 21
Regression model (ln(BMI) = mu + carboh + e, where carboh is
carbohydrate intake in food) within different genotype groups.

| | Unstandardized | | Standardized | | |
|---|---|---|---|---|---|
| | Coefficients | | Coefficients | | |
| | B | Std. Error | Beta | t | Sig. |
| (Constant) | 3.036151 | 0.035271 | | 86.08079 | 0 |
| Age | 0.001751 | 0.000526 | 0.091377 | 3.327731 | 0.000902 |
| HT status | 0.104684 | 0.00869 | 0.329856 | 12.04586 | 1.24E−31 |
| Average weekly | −0.00211 | 0.000705 | −0.08207 | −2.98647 | 0.002879 |
| exercise (hours) | | | | | |
| Dependent Variable: | | | | | |
| BMI_ln | | | | | |

The model was separately used for samples with minor allele present and samples that are homozygous for the major allele.
B = regression coefficient
SE = se of the coefficient
P = P-value of the test statistic
z = (B1 − B2)/sqrt(SE1\hat{ }2 + SE2\hat{ }2)

TABLE 22
Single-SNP associations of SNPs related to the CDKAL1 gene with the carbohydate
intake - BMI interaction.

| SNP | P | HW | MAF | CR | CHR | Position | Gene | GeneID | class |
|---|---|---|---|---|---|---|---|---|---|
| RS16884072 | 5.0772E−06 | 0.127074 | 0.208023 | 1 | 6 | 20763482 | CDKAL1 | 54901 | intron |
| RS736425 | 5.0772E−06 | 0.127074 | 0.208023 | 1 | 6 | 20772291 | CDKAL1 | 54901 | intron |
| RS10484632 | 6.589E−06 | 0.007833 | 0.216239 | 0.998346 | 6 | 20755639 | CDKAL1 | 54901 | intron |
| RS13194407 | 6.6385E−06 | 0.002442 | 0.196443 | 1 | 6 | 20738932 | CDKAL1 | 54901 | intron |

| SNP | B1 | SE1 | P1 | n1 | B2 | SE2 | P2 | n2 | z | P |
|---|---|---|---|---|---|---|---|---|---|---|
| RS16884072 | −0.00178514 | 0.000528 | 0.000796 | 393 | 0.001033 | 0.00032059 | 0.00133622 | 663 | −4.56178 | 5.08E−06 |
| RS736425 | −0.00178514 | 0.000528 | 0.000796 | 393 | 0.001033 | 0.00032059 | 0.00133622 | 663 | −4.56178 | 5.08E−06 |
| RS10484632 | −0.00174339 | 0.000522 | 0.000915 | 403 | 0.001023 | 0.00032304 | 0.0016164 | 651 | −4.50675 | 6.59E−06 |
| RS13194407 | −0.00184241 | 0.000543 | 0.000767 | 375 | 0.000993 | 0.00031776 | 0.0018625 | 681 | −4.50516 | 6.64E−06 |

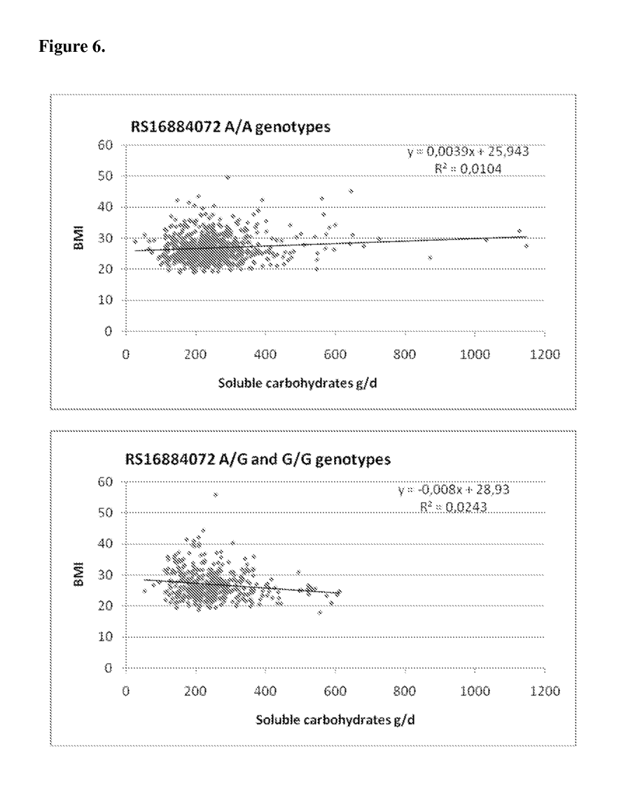

FIG. 6 shows linear regression between soluble carbohydrate intake (g/d) and BMI in RS 16884072 A/G and G/G genotypes. Carbohydrate intake vs BMI in subjects with the

RS16884072 A/A genotype (upper figure) and in the combined group of subjects with A/G or G/G genotype (BMI is used as y value i.e. ordinant instead of ln(BMI) in these figures.)

TABLE 23
The smallest P-values for SNPs in BMI and WHR analyses:

| SNP | MIN_P_BMI | ANALYSIS_2 | MIN_P_WHR | ANALYSIS_WHR |
|---|---|---|---|---|
| RS16884072 | 0.2288 | WOMEN | 0.151045 | P_BIN_VE_MEN_WHR |
| RS736425 | 0.140558 | BINARY BOTH | 0.151045 | P_BIN_VE_MEN_WHR |
| | | GENDERS | | |
| RS10484632 | 0.174052 | BINARY BOTH | 0.085541 | P_BIN_VE_ALL_WHR |
| | | GENDERS | | |
| RS13194407 | 0.1262 | MEN | 0.030742 | P_BIN_VE_MEN_WHR |

D′ and R2 Values for Most Significant Markers in BMI: Carbohydrate Intake×SNP Interaction Analysis

| L1 | L2 | D′ | LOD | r\hat{ }2 | CIlow | CIhi | Dist |
|---|---|---|---|---|---|---|---|
| RS16884072 | RS736425 | 1 | 418.05 | 1 | 0.99 | 1 | 8809 |
| RS16884072 | RS10484632 | 1 | 384.31 | 0.954 | 0.99 | 1 | 7843 |
| RS16884072 | RS13194407 | 0.968 | 318.8 | 0.871 | 0.94 | 0.99 | 24550 |

The most significant markers are in linkage in the Eastern Finnish population

CDKAL1, GenelD: 54901, CDK5 Regulatory Subunit Associated Protein 1-like 1 (mRNA: NM_{—}017774.2; Genomic Reference NC_{—}000006.11)

CDKAL1 gene encodes a 579-residue, 65-kD protein, which function is unknown. However it shares considerable domain and amino acid homology with CDK5RAP1, an inhibitor of CDK5 (cyclin-dependent kinase 5, GeneID: 1020) activation (OMIM). CDK5 has been implicated in the regulation of pancreatic beta cell function through formation of p35/CDK5 complexes that down-regulate insulin expression (Ubeda et al, 2006). CDK5RAP1 is expressed in neuronal tissues, where it inhibits cyclin-dependent kinase 5 (CDK5) activity by binding to the CDK5 regulatory subunit p35. In pancreatic beta cells, CDK5 has been shown to have a role in the loss of beta cell function under glucotoxic conditions. Furthermore, inhibition of the CDK5/p35 complex prevents a decrease of insulin gene expression that results from glucotoxicity. Steinthorsdottir et al. (2007) speculated that CDKAL1 may have a role in the inhibition of the CDK5/p35 complex in pancreatic beta cells similar to that of CDK5RAP1 in neuronal tissue. Reduced expression of CDKAL1 or reduced inhibitory function thus could lead to an impaired response to glucotoxicity.

In genomewide association studies, the Wellcome Trust Case Control Consortium (2007), Diabetes Genetics Initiative of Broad Institute of Harvard and MIT, Lund University, and Novartis I, Zeggini et al. (2007), and Scott et al. (2007) identified association of single-nucleotide polymorphisms (SNPs) within intron 5 of the CDKAL1 gene with susceptibility to type 2 diabetes (OMIM). Barret et al. (2008) have identified the same genomic region associated with Crohn's disease. However, the associated alleles for these two diseases were not correlated. We have also replicated CDKAL1 T2D associated region in our replication study.

Further studies have shown that CDKAL1 diabetes-associated alleles are associated with decreased pancreatic beta-cell function, including decreased beta-cell glucose sensitivity that relates insulin secretion to plasma glucose concentration (Pascoe L et al. 2007). Diabetes-associated variants in CDKAL1 impair insulin secretion and conversion of proinsulin to insulin (Kirchhoff K et al. 2008). Therefore, some CDKAL1 alleles are likely to increase the risk of type 2 diabetes by impairing insulin secretion.

BMI Per Carbohydrate Intake×SNP Interaction Analysis vs T2D Association

The important role of CDKAL1 in glucose induced insulin secretion may explain the result obtained in this analysis (carbohydrate intake). The most significant markers from BMI: Carbohydrate intake×SNP interaction analysis are located near to the region that is shown to be associated with T2D. Haploview image below presents the location and P-values of T2D associated markers that were included into a replication study. Two SNPs that were significantly associated with T2D in the T2D replication are associated in BMI: Carbohydrate intake×SNP interaction analysis (table below). Therefore, it cannot be said whether these two associations are related with each other.

TABLE 24
Association of the strongest T2D related SNPs with the CH-BMI interaction.

| | | | | P for | |
|---|---|---|---|---|---|
| | | | | association | P for |
| | | | | with T2D | BMI*Charbohydrate |
| MARKER | POSITION | GENE | X2 | (replication) | intake interaction |
| RS1569699 | 20787289 | CDKAL1 | 34.81 | 3.62771E−09 | 0.05111152 |
| RS7756992 | 20787688 | CDKAL1 | 34.62 | 3.99569E−09 | 0.04738437 |

References

Barret J C et al. Genome-wide association defines more than 30 distinct suspectibility loci for Crohn's disease Nat Genet 2008; 40:955-962

Diabetes Genetics Initiative of Broad Institute of Harvard and MIT, Lund University, and Novartis Institutes for BioMedical Research:Genome-wide association analysis identifies loci for type 2 diabetes and triglyceride levels. Science 316: 1331-1336, 2007.

Kirchhoff K et al. Polymorphisms in the TCF7L2, CDKAL1 and SLC30A8 genes are associated with impaired proinsulin conversion. Diabetologia. 2008 April;51(4):597-601

Pascoe L et al. Common variants of the novel type 2 diabetes genes CDKAL1 and HHEX/IDE are associated with decreased pancreatic beta-cell function). Diabetes. 2007 December;56(12):3101-4

Scott, L. J.; Mohlke, K. L.; Bonnycastle, L. L.; Willer, C. J.; Li, Y.; Duren, W. L.; Erdos, M. R.; Stringham, H. M.; Chines, P. S.; Jackson, A. U.; Prokunina-Olsson, L.; Ding, C.-J.; and 29 others: A genome-wide association study of type 2 diabetes in Finns detects multiple susceptibility variants. Science 316: 1341-1345, 2007

Steinthorsdottir et al. A variant in CDKAL1 influences insulin response and risk of type 2 diabetes. Nat Genet. 2007 June;39(6):770-5.

M. Ubeda, J. M. Rukstalis, J. F. Habener, Inhibition of cyclin-dependent kinase 5 activity protects pancreatic beta cells from glucotoxicity. J. Biol. Chem. 281, 28858 (2006)

Zeggini, E.; Weedon, M. N.; Lindgren, C. M.; Frayling, T. M.; Elliott, K. S.; Lango, H.; Timpson, N. J.; Perry, J. R. B.; Rayner, N. W.; Freathy, R. M.; Barrett, J. C.; Shields, B.; and 15 others: Replication of genome-wide association signals in UK samples reveals risk loci for type 2 diabetes. Science 316: 1336-1341, 2007.

VWF von Willebrand Factor

BMI Per Carbohydrate Intake×SNP Interaction

Continuous Variable: ln(BMI)

Adjusted for: Age, HT-status, average weekly exercise

TABLE 25
Regression model (ln(BMI) = mu + carboh + e, where carboh is
carbohydrate intake in food) within different genotype groups.

| | Unstandardized | | Standardized | | |
|---|---|---|---|---|---|
| | Coefficients | | Coefficients | | |
| | B | Std. Error | Beta | t | Sig. |
| (Constant) | 3.036151 | 0.035271 | | 86.08079 | 0 |
| Age | 0.001751 | 0.000526 | 0.091377 | 3.327731 | 0.000902 |
| HT status | 0.104684 | 0.00869 | 0.329856 | 12.04586 | 1.24E−31 |
| Average weekly exercise (hours) | −0.00211 | 0.000705 | −0.08207 | −2.98647 | 0.002879 |
| Dependent Variable: BMI_ln | | | | | |

The model was separately used for samples with minor allele present and samples that are homozygous for the major allele.
B = regression coefficient
SE = se of the coefficient
P = P-value of the test statistic
z = (B1 − B2)/sqrt(SE1\hat{ }2 + SE2\hat{ }2)

TABLE 26
Result from BMI per Carbohydrate intake × SNP interaction analysis for
RS17491334, VWF gene.

| SNP | P | HW | MAF | CR | Alleles | CHR | Position | Gene | GeneID | class | Flanking_genes_10K | |
|---|---|---|---|---|---|---|---|---|---|---|---|---|
| RS17491334 | 3.71E−06 | 0.393652 | 0.102151 | 1 | ‘A/G’ | 12 | 5974105 | VWF | 7450 | intron | VWF | 7450 |

| B1 | SE1 | t1 | P1 | n1 | B2 | SE2 | t2 | P2 | n2 | z |
|---|---|---|---|---|---|---|---|---|---|---|
| −0.00242 | 0.000633 | −3.82229 | 0.000178 | 195 | 0.000833 | 0.000305 | 2.726287 | 0.006535 | 861 | −4.62706 |

TABLE 27
Significant P-values for RS17491334 in BMI and WHR analyses

| MARKER | MIN_P_BMI | ANALYSIS_BMI | MIN_P_WHR | ANALYSIS_WHR |
|---|---|---|---|---|
| RS17491334 | 0.4079 | MEN | 0.365562 | P_BIN_VE_WOMEN_WHR |

TABLE 28
P-values for VWF gene in BMI analysis

| MARKER | MIN_P_BMI | ANALYSIS_BMI | CHR | POSITION | CLASS | GENE | GENE_ID | HWE_CONT | MAF | CR |
|---|---|---|---|---|---|---|---|---|---|---|
| RS7955850 | 0.0002766 | MEN | 12 | 6045840 | intron | VWF | 7450 | 0.857624 | 0.074028 | 1 |
| RS216811 | 0.0005105 | WOMEN | 12 | 5985535 | intron | VWF | 7450 | 0.000396 | 0.346761 | 0.995 |

TABLE 29
P-values for VWF gene in WHR analysis

| MARKER | MIN_P_WHR | ANALYSIS_WHR | CHR | POSITION | CLASS |
|---|---|---|---|---|---|
| RS2058473 | 0.005923 | P_VE_WOMEN_WHR | 12 | 5964187 | intron |
| RS216811 | 0.012571 | P_BIN_VE_ALL_WHR | 12 | 5985535 | intron |

| MARKER | GENE | GENE_ID | HWE_CONT | MAF | CR |
|---|---|---|---|---|---|
| RS2058473 | VWF | 7450 | 0.057936 | 0.403146 | 0.999173 |
| RS216811 | VWF | 7450 | 0.000396 | 0.346761 | 0.995864 |

VWF von Willebrand Factor

GeneID: 7450

mRNA: NM_{—}000552.3

Genomic sequence: NC_{—}000012.11

Official Symbol: VWF

Official Full Name von Willebrand factor

Also known as VWD; F8VWF

VWF Literature Related to Body Mass, Insulin Resistance

Meigs J B, O'donnell C J, Tofler G H, Benjamin E J, Fox C S, Lipinska I, Nathan D M, Sullivan L M, D'Agostino R B, Wilson P W. Hemostatic markers of endothelial dysfunction and risk of incident type 2 diabetes: the Framingham Offspring Study. Diabetes. 2006 February;55(2):530-7

“Endothelial dysfunction may precede development of type 2 diabetes. We tested the hypothesis that elevated levels of hemostatic markers of endothelial dysfunction, plasminogen activator inhibitor-1 (PAI-1) antigen, and von Willebrand factor (vWF) antigen predicted incident diabetes independent of other diabetes risk factors. We followed 2,924 Framingham Offspring subjects (54% women, mean age 54 years) without diabetes at baseline (defined by treatment, fasting plasma glucose>or=7 or 2-h postchallenge glucose>or=11.1 mmol/l) over 7 years for new cases of diabetes (treatment or fasting plasma glucose>or=7.0 mmol/l). We used a series of regression models to estimate relative risks for diabetes per interquartile range (IQR) increase in PAI-1 (IQR 16.8 ng/ml) and vWF (IQR 66.8% of control) conditioned on baseline characteristics. Over follow-up, there were 153 new cases of diabetes. Age- and sex-adjusted relative risks of diabetes were 1.55 per IQR for PAI-1 (95% CI 1.41-1.70) and 1.49 for vWF (1.21-1.85). These effects remained after further adjustment for diabetes risk factors (including physical activity; HDL cholesterol, triglyceride, and blood pressure levels; smoking; parental history of diabetes; use of alcohol, nonsteroidal anti-inflammatory drugs, exogenous estrogen, or hypertension therapy; and impaired glucose tolerance), waist circumference, homeostasis model assessment of insulin resistance, and inflammation (assessed by levels of C-reactive protein): the adjusted relative risks were 1.18 per IQR for PAI-1 (1.01-1.37) and 1.39 for vWF (1.09-1.77). We conclude that in this community-based sample, plasma markers of endothelial dysfunction increased risk of incident diabetes independent of other diabetes risk factors including obesity, insulin resistance, and inflammation.”

Mertens I, Van der Planken M, Corthouts B, Van Gaal L F. Is visceral adipose tissue a determinant of von Willebrand factor in overweight and obese premenopausal women? Metabolism. 2006 May;55(5):650-5

“Visceral obesity has been associated with an increased cardiovascular risk. However, the exact mechanisms are not completely clear. In this study we investigated the relationship between von Willebrand factor (vWF) and visceral adipose tissue (VAT) in a group of 181 overweight and obese premenopausal women visiting the weight management clinic of a university hospital. von Willebrand factor antigen (vWF:Ag), plasminogen activator inhibitor 1 (PAI-1) activity, VAT (computed tomography scan), insulin resistance (homeostasis model assessment of insulin resistance), and other anthropometric and metabolic parameters were measured. Subjects with VAT in the highest quintile had significantly higher levels of vWF:Ag (171+/−60 vs 129+/−40%; P=0.001) and PAI-1 (24.7+/−8.5 vs 15.2+/−12.0 AU/mL; P<0.001) compared with subjects in the lowest quintile. After correction for fat mass and homeostasis model assessment of insulin resistance the difference was still significant for vWF:Ag (P=0.046), but not for PAI-1 (P>0.05). Stepwise multiple regression analysis showed VAT and insulin resistance as independent determinants of vWF:Ag, whereas waist circumference, high-density lipoprotein cholesterol, and insulin resistance were independent determinants of PAI-1 activity. In a subgroup of 115 patients, we measured high-sensitivity C-reactive protein and found it to influence the relationship between VAT and vWF:Ag (r=0.16; P=0.088), whereas the relationship with PAI-1 was still significant (r=0.21; P=0.025). The results from this preliminary study suggest a plausible relation between visceral obesity and endothelial activation, possibly mediated by low-grade inflammation”

Garanty-Bogacka B, Syrenicz M, Syrenicz A, Gebala A, Walczak M. Relation of acute-phase reaction and endothelial activation to insulin resistance and adiposity in obese children and adolescents. Neuro Endocrinol Lett. 2005 October;26(5):473-9.

“There is increasing evidence that an ongoing cytokine-induced acute-phase response is closely involved in the pathogenesis of type 2 diabetes and associated complications such as dyslipidemia and atherosclerosis. Garanty-Bogacka et al. (2005) investigated the relationship of inflammation and endothelial activation with insulin resistance in childhood obesity. Two hundred and eleven (122 boys) obese children and adolescents were examined. Fasting levels of ultra-sensitive C-reactive protein (CRP), fibrinogen (FB), interleukin-6 (IL-6), interleukin-lbeta (IL- lbeta), intercellular cell adhesion molecule-1 (ICAM-1), vascular cell adhesion molecule-1 (VCAM-1), von Willebrand factor (vWF), glucose, insulin, and HbA1c were determined. Insulin resistance was assessed by the homeostasis method. HOMA IR correlated significantly with all measures of adiposity as well as with majority of inflammation and endothelial dysfunction markers. After adjustment for age, gender, BMI and fat mass, the correlation with insulin resistance remained significant for CRP, ICAM-1 and von Willebrand factor. There was a trend for association between HOMA IR and IL-6 as well as HOMA IR and fibrinogen. Acute-phase reaction and endothelial activation correlate with insulin resistance in obese youth. It is possible that the cluster of these pro-atherogenic factors may contribute to the accelerated atherosclerosis in obese children”

Weyer C, Yudkin J S, Stehouwer C D, Schalkwijk C G, Pratley R E, Tataranni P A. Humoral markers of inflammation and endothelial dysfunction in relation to adiposity and in vivo insulin action in Pima Indians. Atherosclerosis. 2002 March;161(1):233-42.

“In adults, obesity and IR are associated with higher levels of circulating endothelial dysfunction biomarkers such as soluble intercellular adhesion molecule-1 (sICAM-1) and von Willebrand factor (vWF). Weyer et al (2002) measured fasting plasma concentrations of the inflammatory markers C-reactive protein (CRP), secretory phospholipase A2 (sPLA2) and soluble intercellular adhesion molecule-1 (sICAM-1) and of the endothelial markers E-selectin and von Willebrand factor (vWF) in 32 non-diabetic Pima Indians (18 M/14 F, age 27+/−1 years) in whom percent body fat and insulin-stimulated glucose disposal (M) were assessed by DEXA and a hyperinsulinemic clamp, respectively. CRP, sPLA2, and sICAM-1 were all positively correlated with percent body fat (r=0.71, 0.57, and 0.51, all P<0.01). E-selectin and vWF were not correlated with percent body fat, but were negatively correlated with M (r=−0.65 and −0.46, both P<0.001) and positively correlated with CRP (r=0.46, and 0.33, both P<0.05). These findings indicated that humoral markers of inflammation increase with increasing adiposity in Pima Indians whereas humoral markers of endothelial dysfunction increase primarily in proportion to the degree of insulin resistance and inflammation. Thus, obesity and insulin resistance appear to be associated with low-grade inflammation and endothelial dysfunction, respectively, even in an obesity- and diabetes-prone population with relatively low propensity for atherosclerosis.”

Seligman B G, Biolo A, Polanczyk C A, Gross J L, Clausell N. Increased plasma levels of endothelin 1 and von Willebrand factor in patients with type 2 diabetes and dyslipidemia. Diabetes Care. 2000 September;23(9):1395-400

OBJECTIVE: Endothelial markers endothelin 1 (ET-1) and von Willebrand factor (vWF) were assessed in patients with type 2 diabetes and dyslipidemia and in patients with hypercholesterolemia. RESEARCH DESIGN AND METHODS: In this case-control study, plasma ET-and vWF levels were measured by enzyme-linked immunosorbent assay in 35 normoalbuminuric type 2 diabetic patients with dyslipidemia (56+/−5 years), in 21 nondiabetic patients with hypercholesterolemia (52+/−7 years), and in 19 healthy control subjects (45+/−4 years). All of the individuals were normotensive and nonsmokers. Urinary albumin was measured by immunoturbidimetry. RESULTS: ET-1 levels were higher (P<0.0001) in type 2 diabetic dyslipidemic patients (1.62+/−0.73 pg/ml) than in both nondiabetic hypercholesterolemic patients (0.91+/−0.73 pg/ml) and control subjects (0.69+/−0.25 pg/ml). vWF levels were significantly increased (P=0.02) in type 2 diabetic (185.49+/−72.1%) and hypercholesterolemic (163.29+/−50.7%) patients compared with control subjects (129.70+/−35.2%). In the multiple linear regression analysis. ET-1 was significantly associated (adjusted r2=0.42) with serum triglyceride levels (P<0.001), age (P<0.01), insulin sensitivity index (P<0.02), and albuminuria levels (P<0.04). vWF levels were associated (adjusted r2=0.22) with albuminuria (P<0.001), fibrinogen levels (P<0.02), and BMI (P<0.03). CONCLUSIONS: Compared with hypercholesterolemic patients, type 2 diabetic patients with dyslipidemia have increased levels of ET-1 and vWF which may indicate more pronounced endothelial injury. These findings appear to be related to components of the insulin resistance syndrome.

Summary: The present invention proposes that endothelial dysfunction markers, such as VWF, correlate with obesity and insulin resistance. What is unclear is whether there is any specific metabolic route related to obesity in which VWF could be directly involved or is VWF only a marker of specific metabolic situations.

MS4A2 Membrane-Spanning 4-Domains, Subfamily A, Member 2 (Fc Fragment of IgE, High Affinity I, Receptor for; Beta Polypeptide)

Official Symbol: MS4A2

Official Full Name: membrane-spanning 4-domains, subfamily A, member 2 (Fc fragment of IgE, high affinity I, receptor for; beta polypeptide)

Also known as: APY; IGEL; IGER; ATOPY; FCERI; IGHER; MS4A1; FCER1B

GeneID: 2206

mRNA: NM_{—}000139.3

Genomic Sequence: NC_{—}000011.9

Summary: The allergic response involves the binding of allergen to receptor-bound IgE followed by cell activation and the release of mediators responsible for the manifestations of allergy. The IgE-receptor, a tetramer composed of an alpha, beta, and 2 disulfide-linked gamma chains, is found on the surface of mast cells and basophils. This gene encodes the beta subunit of the high affinity IgE receptor which is a member of the membrane-spanning 4A gene family. Members of this nascent protein family are characterized by common structural features and similar intron/exon splice boundaries and display unique expression patterns among hematopoietic cells and nonlymphoid tissues. This family member is localized to 1 1q12, among a cluster of family members. (Entrez)

Function: Binds to the Fc region of immunoglobulins epsilon. High affinity receptor. Responsible for initiating the allergic response. Binding of allergen to receptor-bound IgE leads to cell activation and the release of mediators (such as histamine) responsible for the manifestations of allergy. The same receptor also induces the secretion of important lymphokines (GeneCards)

References

Donnadieu, E.; Jouvin, M.-H.; Rana, S.; Moffatt, M. F.; Mockford, E. H.; Cookson, W. O.; Kinet, J.-P. :Competing functions encoded in the allergy-associated Fc-epsilon-RI-beta gene. Immunity 18: 665-674, 2003.

Foister-Hoist, R.; Moises, H. W.; Yang, L.; Fritsch, W.; Weissenbach, J.; Christophers, E. Linkage between atopy and the IgE high-affinity receptor gene at 11q13 in atopic dermatitis families. Hum. Genet. 102: 236-239, 1998.

Hill, M. R.; Cookson, W. O. C. M. A new variant of the beta subunit of the high-affinity receptor for immunoglobulin E (Fc-epsilon-RI-beta E237G): associations with measures of atopy and bronchial hyper-responsiveness. Hum. Molec. Genet. 5: 959-962, 1996.

Hizawa, N.; Yamaguchi, E.; Furuya, K.; Ohnuma, N.; Kodama, N.; Kojima, J.; Ohe, M.; Kawakami, Y. Association between high serum total IgE levels and D11S97 on chromosome 11q13 in Japanese subjects. J. Med. Genet. 32: 363-369, 1995.

Kuster, H.; Zhang, L.; Brini, A. T.; MacGlashan, D. W. J.; Kinet, J.-P. The gene and cDNA for the human high affinity immunoglobulin E receptor beta chain and expression of the complete human receptor. J. Biol. Chem. 267: 12782-12787, 1992.

Sandford, A. J.; Shirakawa, T.; Moffatt, M. F.; Daniels, S. E.; Ra, C.; Faux, J. A.; Young, R. P.; Nakamura, Y.; Lathrop, G. M.; Cookson, W. O. C. M.; Hopkin, J. M. Localisation of atopy and beta subunit of high-affinity IgE receptor (FCER1) on chromosome 11q. Lancet 341: 332-334, 1993.

Shirakawa, T.; Li, A.; Dubowitz, M.; Dekker, J. W.; Shaw, A. E.; Faux, J. A.; Ra, C.; Cookson, W. O. C. M.; Hopkin, J. M. Association between atopy and variants of the beta subunit of the high-affinity immunoglobulin E receptor. Nature Genet. 7: 125-130, 1994.

Traherne, J. A.; Hill, M. R.; Hysi, P.; D′Amato, M.; Broxholme, J.; Mott, R.; Moffatt, M. F.; Cookson, W. O. C. M. LD mapping of maternally and non-maternally derived alleles and atopy in Fc-epsilon-RI-beta. Hum. Molec. Genet. 12: 2577-2585, 2003.

NAALADL2, N-Acetylated Alpha-Linked Acidic Dipeptidase-Like 2

GeneID: 254827

mRNA: NM_{—}207015.2

Genomic Sequence: NC 000003.11

Official Symbol: NAALADL2

Official Full Name: N-acetylated alpha-linked acidic dipeptidase-like 2

Function: not known

Domain Descriptions: PA_hNAALADL2_like: Protease-associated domain containing proteins like human N-acetylated alpha-linked acidic dipeptidase-like 2 protein (hNAALADL2). This group contains various PA domain-containing proteins similar to hNAALADL2. The function of hNAALADL2 is unknown. This gene has been mapped to a chromosomal region associated with Cornelia de Lange syndrome. The significance of the PA domain to hNAALADL2 has not been ascertained. It may be a protein-protein interaction domain. At peptidase active sites, the PA domain may participate in substrate binding and/or promoting conformational changes, which influence the stability and accessibility of the site to substrate.

TFR_dimer; Transferrin receptor-like dimerisation domain. This domain is involved in dimerisation of the transferrin receptor as shown in its crystal structure.

M20_dimer Super-family; Peptidase dimerisation domain. This domain consists of 4 beta strands and two alpha helices which make up the dimerisation surface of members of the M20 family of peptidases. This family includes a range of zinc metallopeptidases belonging to several families in the peptidase classification. Family M20 are Glutamate carboxypeptidases. Peptidase family M25 contains X-His dipeptidases.

References

1. Tonkin, E. T.; Smith, M.; Eichhorn, P.; Jones, S.; Imamwerdi, B.; Lindsay, S.; Jackson, M.; Wang, T.-J.; Ireland, M.; Burn, J.; Krantz, I. D.; Carr, P.; Strachan, T.:

A giant novel gene undergoing extensive alternative splicing is severed by a Cornelia de Lange-associated translocation breakpoint at 3q26.3. Hum. Genet. 115: 139-148, 2004.

All publications, patents, patent applications, Gene IDs, and accession numbers for nucleic acid or amino acid sequences cited herein are hereby incorporated by reference in their entirety for all purposes to the same extent as if each individual publication, patent, patent application, nucleic acid or amino acid sequence were specifically and individually indicated to be so incorporated by reference (i.e. as if the publications and sequences were disclosed as such in the specification).

1-80. (canceled)

81. A method for risk assessment, diagnosis or prognosis of obesity or type 2 diabetes (T2D) in a mammalian subject comprising:
a) providing a biological sample taken from the subject;
b) detecting one or more T2D and/or obesity associated genetic markers in said sample, wherein the genetic markers are related to SUCLA2 gene, and;
c) comparing the genetic marker data from the subject to genetic marker data from healthy and diseased people to make risk assessment, diagnosis or prognosis of obesity or T2D.

82. The method according to claim 81, wherein the genetic marker is SNP marker RS12873870 in SUCLA2 gene.

83. A test kit for risk assessment, diagnosis or prognosis of obesity or T2D comprising:
a) reagents, materials and protocols for assessing type and/or level of one or more T2D and/or obesity phenotype associated genetic markers in a biological sample, wherein the genetic markers are related to SUCLA2 gene, and;
b) instructions and software for comparing the genetic marker data from a subject to genetic marker data from healthy and diseased people to make risk assessment, diagnosis or prognosis of obesity or T2D.

84. The kit according to claim 83, wherein the genetic marker is SNP marker RS12873870 in SUCLA2 gene.

85. A method for screening agents for preventing or treating obesity or T2D in a mammal comprising determining the effect of an agent either on a metabolic pathway related to a polypeptide or a RNA molecule encoded by SUCLA2 gene in living cells; wherein an agent altering activity of the metabolic pathway is considered useful in prevention or treatment of obesity or T2D.

86. Method for monitoring a risk of an individual to become obese comprising a step of measuring the urinary excretion of a Krebs cycle metabolite dependent on SUCLA2 gene activity, wherein the metabolite of the SUCLA2 gene is urinary methylmalonic acid, succinate (succinic acid), fumarate (fumaric acid) or succinyl-CoA synthetase activity.

87. A method for risk assessment, diagnosis or prognosis of obesity, type 2 diabetes (T2D) or a T2D related condition in a mammalian subject comprising:
a) providing a biological sample taken from the subject;
b) detecting one or more T2D and/or obesity or related phenotype associated biomarkers in said sample, wherein the biomarkers are related to one or more genes selected from the group consisting of SUCLA2, KLF4, MS4A2, ANO5, NELL1, DNAH11, RNF216, VGLL3, CDKAL1, VWF, NAALADL2, HSL, PLIN1, and PRKACA or said biomarkers are related to one or more polypeptides encoded by said genes, and;
c) comparing the biomarker data from the subject to biomarker data from healthy and diseased people to make risk assessment, diagnosis or prognosis of obesity, T2D or a T2D related condition.

88. The method according to claim 87, wherein said obesity or T2D related condition comprises glucose intolerance, insulin resistance, metabolic syndrome, obesity, a microvascular complication such as retinopathy, nephropathy or neuropathy, or a macrovascular complication such as coronary heart disease, cerebrovascular disease, congestive heart failure, claudication or other clinical manifestation of atherosclerosis or arteriosclerosis.

89. The method according to claim 87, wherein at least one biomarker is a metabolite of a polypeptide encoded by a gene selected from the group consisting of SUCLA2, KLF4, MS4A2, ANO5, NELL1, DNAH11, RNF216, VGLL3, CDKAL1, VWF, NAALADL2, HSL, PLIN1, and PRKACA.

90. The method according to claim 89, wherein the metabolite of gene SUCLA2 is selected from the group consisting of plasma, serum or blood cell or urinary methylmalonic acid, succinate (succinic acid) and fumarate (fumaric acid).

91. A test kit for risk assessment, diagnosis or prognosis of obesity, T2D or a T2D related condition comprising:
a) reagents, materials and protocols for assessing type and/or level of one or more T2D and/or obesity phenotype associated biomarkers in a biological sample, wherein the biomarkers are related to one or more genes selected from the group consisting of SUCLA2, KLF4, MS4A2, ANO5, NELL1, DNAH11, RNF216, VGLL3, CDKAL1, VWF, NAALADL2, HSL, PLIN1, and PRKACA or said biomarkers are related to one or more polypeptides encoded by said genes, and;
b) instructions and software for comparing the biomarker data from a subject to biomarker data from healthy and diseased people to make risk assessment, diagnosis or prognosis of obesity, T2D or a T2D related condition.

92. A method for screening agents for preventing or treating obesity, T2D or a T2D related condition in a mammal comprising determining the effect of an agent either on a metabolic pathway related to a polypeptide or a RNA molecule encoded by a T2D and/or obesity associated gene selected from the group consisting of SUCLA2, KLF4, MS4A2, ANO5, NELL1, DNAH11, RNF216, VGLL3, CDKAL1, VWF, NAALADL2, HSL, PLIN1, and PRKACA in living cells; wherein an agent altering activity of a metabolic pathway is considered useful in prevention or treatment of obesity, T2D or a T2D related condition.

93. The method according to claim 92, wherein said agent is administered to a model system or organism, and wherein an agent altering or modulating expression, biological activity or function of a T2D and/or obesity associated gene selected from the group consisting of SUCLA2, KLF4, MS4A2, ANO5, NELL1, DNAH11, RNF216, VGLL3, CDKAL1, VWF, NAALADL2, HSL, PLIN1, and PRKACA or it's encoded polypeptide is considered useful in prevention or treatment of obesity, T2D or a T2D related condition.

94. Method for monitoring energy efficiency, energy consumption and physical activity of an individual or a risk of an individual to become obese comprising a step of measuring the urinary excretion of a Krebs cycle metabolite dependent on SUCLA2 gene activity.

95. The method according to claim 94, wherein said metabolite is methylmalonate or methylcitrate.

96. Method for monitoring or assessing energy intolerance or energy efficiency of an individual comprising a step of calculating the ratio of body mass index, BMI, and/or waist-hip ratio, WHR, to dietary energy intake, wherein high ratio of BMI and/or WHR to dietary energy intake denotes energy intolerance, i.e. BMI/WHR tends to rise easier or at a lower energy intake levels.

97. Recombinant HSL or analogs of HSL for use in the treatment of obesity, type 2 diabetes (T2D) or a T2D related condition.

98. Recombinant perilipin A or analogs of perilipin A or cAMP-dependent protein kinase for use in the treatment of obesity, type 2 diabetes (T2D) or a T2D related condition.

99. Method for treatment of obesity, type 2 diabetes (T2D) or a T2D related condition, wherein a pharmaceutically effective amount of recombinant HSL, analogs of HSL, recombinant perilipin A, analogs of perilipin A or cAMP-dependent protein kinase is administered to a patient in need of such treatment.
